SEQUENCE LISTING (5 sequences)

SEQ ID NO: 1 - protein, 12 aa, Homo sapiens
LLEFYLAMPFAT

SEQ ID NO: 2 - protein, 12 aa, Homo sapiens
VLLKEFTVSGNI

SEQ ID NO: 3 - protein, 12 aa, Homo sapiens
RQLQLSISSCLQ

SEQ ID NO: 4 - protein, 14 aa, Homo sapiens
SLLMWITQCFLPVF

SEQ ID NO: 5 - protein, 15 aa, Homo sapiens
ASRELERFAVNPGLL

(19) United States
(12) Patent Application Publication
(10) Pub. No.: US 2009/0136446 A1
(43) Pub. Date: May 28, 2009

(54) INDUCTION OF REGULATORY T CELL-RESISTANT HELPER CD4+ T CELLS

(75) Inventors: HIROYOSHI NISHIKAWA, MIE (JP); HIROSHI SHIKU, MIE (JP); GERD RITTER, NEW YORK, NY (US); LLOYD J. OLD, NEW YORK, NY (US); SACHA GNJATIC, NEW YORK, NY (US)

Correspondence Address: WOLF GREENFIELD & SACKS, P.C., 600 ATLANTIC AVENUE, BOSTON, MA, 02210-2206, US

(73) Assignee: Ludwig Institute for Cancer Research Ltd., New York, NY (US)

(21) Appl. No.: 12/262,831

(22) Filed: Oct. 31, 2008

Related U.S. Application Data
(60) Provisional application No. 61/001,626, filed on Nov. 2, 2007.

(51) Int. Cl. A61K 38/20; C12N 5/06; A61K 39/00; A61K 35/12

(52) U.S. Cl. 424/85.2; 435/375; 424/184.1; 424/277.1; 424/93.71

(57) ABSTRACT

The invention relates to stimulation of immune responses against antigen(s) and overcoming regulatory T cell suppression of such immune responses against antigen(s).

### RELATED APPLICATION

This application claims the benefit under 35 U.S.C. 119(e) of U.S. provisional application Ser. No. 61/001,626, filed Nov. 2, 2007, the entire contents of which are incorporated by reference herein.

### FIELD OF THE INVENTION

The invention relates to stimulation of immune responses against antigen(s) and overcoming regulatory T cell suppression of such immune responses against antigen(s).

### BACKGROUND OF THE INVENTION

With the molecular identification of tumor specific antigens, a number of cancer vaccine trials targeting these antigens in the form of synthetic peptide epitopes have been attempted. Specific immune responses were observed in some clinical trials, however, only a minority of treated patients experienced clinical responses. Because of the weak clinical effectiveness of current cancer vaccine trials, many investigators are trying to discover more immunogenic antigens, new effective adjuvants, formulations, vectors or vaccination methods (Jager, E., et al., Int. J. Cancer. 106:817-20, 2003; Belardelli, F., et al., Cancer Res. 64:6827-30, 2004; Rosenberg, S. A., et al., Nat. Med. 10:909-15, 2004). Another obstacle for cancer vaccine trials is that patient enrollment is often limited by the indispensable requirement for antigen expression in cancer cells. Most attractive tumor specific antigens discovered so far have only a limited expression frequency in cancer, thus preventing many patients to meet adequate eligibility criteria.

NY-ESO-1 is a germ cell protein that is often expressed by cancer cells, but not normal somatic cells (Chen, Y. T. et al., Proc. Natl. Acad. Sci. USA. 94:1914-8, 1997). It was discovered by serological identification of antigens by recombinant expression cloning (SEREX) using the serum of an esophageal cancer patient (Chen, Y. T. et al., Proc. Natl. Acad. Sci. USA. 94:1914-8, 1997; Sahin, U. et al., Proc. Natl. Acad. Sci. USA. 92:11810-3, 1995). The frequent finding of humoral and cellular immune responses against this antigen in cancer patients with NY-ESO-1 expressing tumors makes it one of the most immunogenic human tumor antigens (Jager, E. et al., J. Exp. Med. 187:265-70, 1998). However, the frequency of NY-ESO-1 expression in melanoma, lung, breast, ovarian, and bladder cancers is only 20-30% and often heterogeneous (Chen, Y. T. et al., Proc. Natl. Acad. Sci. USA. 94: 1914-8, 1997; Jungbluth, A. A. et al., Int. J. Cancer. 92:856-60, 2001).

Recent reports found that toll like receptor signals were important not only for eliciting immune responses but also for blocking suppressive activity by regulatory T cells, and recombinant viral and bacterial vectors have attracted attention for potentially providing necessary danger signals in vaccine vectors (Belardelli, F., et al., Cancer Res. 64:6827-30, 2004; Rosenberg, S. A., et al., Nat. Med. 10:909-15, 2004; Pasare, C. & Medzhitov, R., Science. 299:1033-6, 2003; Yang, Y., et al., Nat. Immunol. 5:508-15, 2004). One of the most promising candidates among bacterial vectors is Salmonella enterica serovar Typhimurium (S. typhimurium) (Russmann, H. et al., Science. 281:565-8, 1998; Shams, H., et al., Vaccine. 20:577-85, 2001; Evans, D. T. et al., J. Virol. 77:2400-9, 2003). The simplicity of its administration, the ease of its genetic manipulation, and the availability of several virulence-attenuating mutations have made S. typhimurium a very versatile antigen delivery platform (Galán, J. E. et al., Gene 94:29-35, 1990; Russman et al., 1998). Contact of Salmonella typhimurium with host cells causes activation of specialized protein secretion system, termed type III, that delivers a set of bacterial cytosolic proteins to the host cell cytosol without requiring bacterial uptake by target cells (Russmann, H. et al., Science. 281: 565-8, 1998; Stebbins, C. E. & Galan, J. E., Nature. 414:77-81, 2001; U.S. Pat. No. 6,306,387; WO/2007/044406).

Although advances in the treatment of cancer have been made, there still exists a need for improved methods of treating cancer. In particular, there exists a need for improved cancer vaccines that can be used therapeutically and/or prophylactically.

### SUMMARY OF THE INVENTION

The present invention addresses this need by providing methods for treating cancer using avirulent bacteria having a type III secretion system to deliver an antigen to a cell. In addition, the present invention discloses strategies for controlling Tregs in the cancer vaccine field.

We have found that in healthy donors and melanoma patients without NY-ESO-1 spontaneous immunity, S. typhimurium-NY-ESO-1 elicits CD4+ T helper 1 (Th1) cells in vitro recognizing naturally processed antigen from these high-avidity NY-ESO-1-specific naïve precursors. In contrast to peptide stimulation, induction of specific Th1 cells with S. typhimurium-NY-ESO-1 did not require in vitro depletion of CD4+CD25+ Tregs and this prevailing effect was partially blocked by disruption of interleukin-6 or glucocorticoid-induced TNF receptor (GITR) signals. Furthermore, S. typhimurium-induced Th1 cells had higher GITR expression than peptide-induced Th1 cells and were resistant to suppression by CD4+CD25+ Tregs in a GITR dependent fashion. Thus, S. typhimurium-NY-ESO-1 induces antigen-specific T cell responses that are resistant to suppression by CD4+CD25+ Tregs.

In aspects of the invention, methods for producing antigen-specific CD4+ T cells in the presence of CD4+CD25+ T regulatory cells are provided. The methods include contacting a population of CD4+ precursor cells with antigen presenting cells, wherein the antigen presenting cells have been infected with or exposed to avirulent bacteria comprising a type III secretion system and a nucleic acid encoding a fusion protein that comprises a polypeptide antigen or an immunogenic fragment thereof fused to a polypeptide that is delivered by the type III secretion system of the avirulent bacteria, wherein the population of CD4+ precursor cells is not depleted of CD4+CD25+ T regulatory cells, and whereby the fusion protein stimulates production of antigen-specific CD4+ T cells that are specific for the polypeptide antigen or the immunogenic fragment thereof. In some embodiments, the step of contacting a population of CD4+ precursor cells comprises administering the avirulent bacteria to a subject in need of such treatment, in amounts of each that are effective to stimulate production of antigen-specific CD4+ T cells. In certain embodiments, the CD4+ precursor cells are peripheral blood mononuclear cells. In certain other embodiments, the methods include isolating the antigen-specific CD4+ T cells.

In some embodiments, the polypeptide antigen or immunogenic fragment thereof is a tumor antigen protein or an immunogenic fragment thereof. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent bacteria are S. typhimurium pSB2470.

In some embodiments, more than one polypeptide antigen or immunogenic fragment thereof is encoded.

In some embodiments, the antigen-specific CD4+ T cells are T helper 1 (Th1) cells.

In some embodiments, the antigen-specific CD4+ T cells are resistant to anti-proliferative effects of CD4+CD25+ T regulatory cells.

In some embodiments, the antigen-specific CD4+ T cells are activated high-avidity antigen-specific CD4+ T cell precursors from a CD45RA+ population.

In some embodiments, methods for passive immunization are provided. The methods include administering to a subject in need of such treatment a population of antigen-specific CD4+ T cells produced in the presence of CD4+CD25+ T regulatory cells by any of the foregoing methods.

In other aspects of the invention, methods for producing antigen-specific CD4+ T cells in the presence of CD4+CD25+ T regulatory cells are provided. The methods include contacting a population of CD4+ precursor cells with antigen presenting cells, wherein the antigen presenting cells have been infected with avirulent bacteria comprising a type III secretion system and contacted with a polypeptide antigen or an immunogenic fragment thereof, wherein the population of CD4+ precursor cells is not depleted of CD4+CD25+ T regulatory cells, and whereby the polypeptide antigen or the immunogenic fragment thereof stimulates production of antigen-specific CD4+ T cells specific for the polypeptide antigen or the immunogenic fragment thereof.

In some embodiments, the step of contacting a population of CD4+ precursor cells comprises administering the avirulent bacteria and the polypeptide antigen or the immunogenic fragment thereof to a subject in need of such treatment, in amounts of each that are effective to stimulate production of antigen-specific CD4+ T cells. In certain embodiments, the CD4+ precursor cells are peripheral blood mononuclear cells. In certain other embodiments, the methods further comprise isolating the antigen-specific CD4+ T cells.

In some embodiments, the polypeptide antigen or immunogenic fragment thereof is a tumor antigen protein or an immunogenic fragment thereof. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent bacteria are S. typhimurium pSB2470.

In some embodiments, the antigen presenting cells are contacted with more than one polypeptide antigen or immunogenic fragment thereof.

In some embodiments, the antigen-specific CD4+ T cells are T helper 1 (Th1) cells.

In some embodiments, the antigen-specific CD4+ T cells are resistant to anti-proliferative effects of CD4+CD25+ T regulatory cells.

In some embodiments, the antigen-specific CD4+ T cells are activated high-avidity antigen-specific CD4+ T cell precursors from a CD45RA+ population. In some embodiments, methods for passive immunization are provided. The methods include administering to a subject in need of such treatment a population of antigen-specific CD4+ T cells produced in the presence of CD4+CD25+ T regulatory cells by any of the foregoing methods.

In further aspects of the invention, methods for producing antigen-specific CD4+ T cells in the presence of CD4+CD25+ T regulatory cells are provided. The methods include contacting a population of CD4+ precursor cells with antigen presenting cells, wherein the antigen presenting cells have been contacted with glucocorticoid-induced TNF receptor ligand (GITRL), and a polypeptide antigen or an immunogenic fragment thereof, wherein the population of CD4+ precursor cells is not depleted of CD4+CD25+ T regulatory cells, and whereby the polypeptide antigen or the immunogenic fragment thereof stimulates production of antigen-specific CD4+ T cells specific for the polypeptide antigen or the immunogenic fragment thereof. In some embodiments, the step of contacting a population of CD4+ precursor cells comprises administering the GITRL and the polypeptide antigen or the immunogenic fragment thereof to a subject in need of such treatment, in amounts of each that are effective to stimulate production of antigen-specific CD4+ T cells. In certain embodiments, the CD4+ precursor cells are peripheral blood mononuclear cells. In certain other embodiments, the methods include isolating the antigen-specific CD4+ T cells.

In some embodiments, the polypeptide antigen or immunogenic fragment thereof is a tumor antigen protein or an immunogenic fragment thereof. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the antigen presenting cells are contacted with more than one polypeptide antigen or immunogenic fragment thereof.

In some embodiments, the antigen-specific CD4+ T cells are T helper 1 (Th1) cells.

In some embodiments, the methods include contacting the antigen presenting cells with interleukin-6 (IL-6).

In some embodiments, the GITRL is a recombinant GITRL-Fc fusion protein.

In some embodiments, the antigen-specific CD4+ T cells are resistant to anti-proliferative effects of CD4+CD25+ T regulatory cells.

In some embodiments, the antigen-specific CD4+ T cells are activated high-avidity antigen-specific CD4+ T cell precursors from a CD45RA+ population.

In some embodiments, methods for passive immunization are provided. The methods include administering to a subject in need of such treatment a population of antigen-specific CD4+ T cells produced in the presence of CD4+CD25+ T regulatory cells by any of the foregoing methods.

In still other aspects of the invention, methods for preparing antigen presenting cells from peripheral blood mononuclear cells are provided. The methods include obtaining peripheral blood mononuclear cells (PBMCs) from a subject, wherein the PBMCs are not depleted of CD4+CD25+ T regulatory cells, contacting the PBMCs with a recombinant avirulent bacteria comprising a type III secretion system that expresses by the type III secretion system an antigen, culturing the contacted PBMCs, and isolating antigen presenting cells.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent S. typhimurium bacteria are S. typhimurium pSB2470.

In some embodiments, the antigen is a tumor antigen. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, an isolated population of antigen presenting cells is provided, wherein the antigen presenting cells are prepared by any of the foregoing methods.

In other aspects of the invention, methods for preparing antigen-specific T cells are provided. The methods include obtaining peripheral blood mononuclear cells (PBMCs) from a subject, contacting the PBMCs with antigen presenting cells, prepared by any of the foregoing methods, culturing the contacted PBMCs, and isolating antigen-specific T cells from the PBMCs. In certain embodiments, the antigen-specific T cells are CD4⁺ T cells.

In some embodiments, an isolated population of antigen-specific T cells is provided, wherein the antigen-specific T cells are prepared by any of the foregoing methods. In some embodiments, an isolated population of antigen-specific T cells is provided, wherein the antigen-specific T cells are prepared by any of the foregoing methods, and wherein the T cells are CD4⁺ T cells.

In some embodiments, methods for passive immunization are provided. The methods include administering to a subject in need of such treatment any of the foregoing populations of antigen-specific T cells.

In additional aspects of the invention, methods for preparing antigen presenting cells from peripheral blood mononuclear cells are provided. The methods include obtaining peripheral blood mononuclear cells (PBMCs) from a subject, wherein the PBMCs are not depleted of CD4+CD25+ T regulatory cells, contacting the PBMCs with a recombinant avirulent bacteria comprising a type III secretion system, and an antigen, culturing the contacted PBMCs, and isolating antigen presenting cells.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. Yet, in other embodiments, avirulent S. typhimurium bacteria are S. typhimurium pSB2470.

In some embodiments, the antigen is a tumor antigen. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, an isolated population of antigen presenting cells is provided, wherein antigen presenting cells are prepared by any of the foregoing methods.

In some embodiments, a method for preparing antigen-specific T cells is provided. The methods include obtaining peripheral blood mononuclear cells (PBMCs) from a subject, contacting the PBMCs with any of the foregoing antigen presenting cells, culturing the contacted PBMCs, and isolating antigen-specific T cells from the PBMCs. In certain embodiments, the antigen-specific T cells are CD4⁺ T cells. In some embodiments, an isolated population of antigen-specific T cells is provided, wherein the antigen-specific T cells are prepared by any of the foregoing methods.

In some embodiments, an isolated population of antigen-specific T cells is provided, wherein the antigen-specific T cells are prepared by any of the foregoing methods, and wherein the T cells are CD4⁺ T cells.

In some embodiments, methods for passive immunization are provided. The methods include administering to a subject in need of such treatment any of the foregoing populations of antigen-specific T cells.

In further aspects of the invention, methods for preparing antigen presenting cells from peripheral blood mononuclear cells are provided. The methods include obtaining peripheral blood mononuclear cells (PBMCs) from a subject, wherein the PBMCs are not depleted of CD4+CD25+ T regulatory cells, contacting the PBMCs with a glucocorticoid-induced TNF receptor ligand (GITRL) and optionally with interleukin-6 (IL-6), and an antigen, culturing the contacted PBMCs, and isolating antigen presenting cells. In certain embodiments, the GITRL is a recombinant GITRL-Fc fusion protein.

In some embodiments, the antigen is a tumor antigen. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, an isolated population of antigen presenting cells is provided, wherein the antigen presenting cells are prepared by any of the foregoing methods.

In some embodiments, a method for preparing antigen-specific T cells is provided. The method includes obtaining peripheral blood mononuclear cells (PBMCs) from a subject, contacting the PBMCs with any of the foregoing antigen presenting cells, culturing the contacted PBMCs, and isolating antigen-specific T cells from the PBMCs. In certain embodiments, the antigen-specific T cells are CD4⁺ T cells. In some embodiments, an isolated population of antigen-specific T cells is provided, wherein the antigen-specific T cells are prepared by any of the foregoing methods. In some embodiments, an isolated population of antigen-specific T cells is provided, wherein the antigen-specific T cells are prepared by any of the foregoing methods, and wherein the T cells are CD4⁺ T cells.

In some embodiments, methods for passive immunization are provided. The methods include administering to a subject in need of such treatment any of the foregoing populations of antigen-specific T cells.

In aspects of the invention, methods for predicting whether a subject will respond to vaccination with avirulent bacteria are provided. The methods include a type III secretion system and a nucleic acid encoding a fusion protein that comprises a polypeptide antigen or an immunogenic fragment thereof fused to a polypeptide that is delivered by the type III secretion system of the avirulent bacteria and elicits antigen-specific CD4⁺ T cells. The methods involve obtaining a biological sample from the subject, assaying the sample to determine the presence in the sample of high-avidity antigen-specific naïve CD4⁺ T cell precursors, wherein the presence of the high-avidity antigen-specific naïve CD4⁺ T cell precursors indicates that the subject will respond to vaccination with the avirulent bacteria and elicit antigen-specific CD4⁺ T cells.

In some embodiments, the polypeptide antigen or immunogenic fragment thereof is a tumor antigen protein or an immunogenic fragment thereof. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent bacteria are S. typhimurium pSB2470.

In further aspects of the invention, methods for upregulating expression of glucocorticoid-induced TNF receptor (GITRL) on antigen presenting cells is provided. The methods include producing antigen presenting cells by contacting antigen presenting cell precursors with antigen presenting cells infected with avirulent bacteria comprising a type III secretion system, wherein the antigen presenting cells have upregulated expression of GITRL as compared with antigen presenting cells produced by contacting antigen presenting cell precursors with antigen presenting cells contacted with peptide antigens.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent bacteria are S. typhimurium pSB2470.

In additional aspects of the invention, methods for upregulating expression of glucocorticoid-induced TNF receptor ligand (GITRL) on antigen presenting cells are provided. The methods include infecting antigen presenting cells or antigen presenting cell precursors with avirulent bacteria comprising a type III secretion system, wherein the antigen presenting cells have upregulated expression of GITRL as compared with antigen presenting cells contacted with a polypeptide antigen.

In some embodiments, the avirulent bacteria are Salmonella spp., Yersinia spp., Bordetella spp., Escherichia coli, Shigella spp., Burkholderia mallei, Burkholderia pseudomallei or Pseudomonas aeruginosa. In certain embodiments, the avirulent bacteria are Salmonella enterica. In certain other embodiments, the avirulent S. enterica bacteria are S. typhimurium. In other embodiments, the avirulent bacteria are S. typhimurium pSB2470.

In yet other aspects of the invention, kits for immunization are provided. The kits include a first container containing one or more doses of a vaccine against an antigen and a second container containing an avirulent bacteria comprising a type III secretion system that does not express the antigen by the type III secretion system.

In some embodiments, the antigen is not a tumor antigen. In certain embodiments, the antigen causes mumps, measles, rubella, chicken pox, influenza, diphtheria, tetanus, pertussis, hepatitis A, hepatitis B, bacterial meningitis (Haemophilus influenzae type b), polio or Streptococcus pneumoniae infection (invasive pneumococcal disease).

In some embodiments, the antigen is a tumor antigen. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the vaccine against the antigen is a DNA vaccine vector encoding the antigen.

In further aspects of the invention, kits for immunization are provided. The kits include a first container containing one or more doses of a vaccine against an antigen, a second container containing an amount of glucocorticoid-induced TNF receptor ligand (GITRL), and a third container containing an amount of interleukin-6 (IL-6).

In some embodiments, the antigen is not a tumor antigen. In certain embodiments, the antigen causes mumps, measles, rubella, chicken pox, influenza, diphtheria, tetanus, pertussis, hepatitis A, hepatitis B, bacterial meningitis (Haemophilus influenzae type b), polio or Streptococcus pneumoniae infection (invasive pneumococcal disease).

In some embodiments, the antigen is a tumor antigen. In certain embodiments, the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

In some embodiments, the vaccine against the antigen is a DNA vaccine vector encoding the antigen.

In some embodiments, the GITRL is a recombinant GITRL-Fc fusion protein.

These and other objects of the invention will be described in further detail in connection with the detailed description of the invention.

### BRIEF DESCRIPTION OF THE DRAWINGS

FIG. 1. NY-ESO-1-specific CD4⁺ T cells are elicited by S. typhimurium-NY-ESO-1 without the need for CD4⁺CD25⁺ T cell depletion.

Presence of NY-ESO-1 peptide-specific CD4⁺ Th1 cells in whole CD4⁺ T cells and CD4⁺CD25⁻ T cells was analyzed by ELISPOT assays following a 15 to 20 day culture with APC pulsed with indicated NY-ESO-1 peptides (except NC155; NY-ESO-1 157-170) or infected with S. typhimurium-NY-ESO-1 (indicated “S. typh.-NY-ESO-1”) or S. typhimurium control strain (indicated “S. typh.-control strain”). Responses were analyzed by specific IFN-γ secretion for recognition of autologous T-APC pulsed with indicated peptide (except NC155; NY-ESO-1 157-170 (grey column), or HIV peptide (open column)), in 4 healthy donors (A) and in 4 patients (B) with NY-ESO-1 expressing tumors but without NY-ESO-1 antibody. These experiments were performed independently at least twice with similar results. Data are expressed as mean±SD.

FIG. 2. S. typhimurium-induced NY-ESO-1-specific CD4⁺ Th1 cells are able to recognize naturally processed NY-ESO-1 protein.

(A) Whole CD4⁺ T cells or CD4⁺ CD25⁻ T cells of NC235 or NW681 were isolated from PBMC and cultured with APC infected with S. typhimurium-NY-ESO-1 or pulsed with indicated NY-ESO-1 peptide, respectively. Fifteen to 20 days later, the capacity of elicited NY-ESO-1-specific Th1 cells to recognize naturally processed NY-ESO-1 protein was analyzed by ELISPOT assay using NY-ESO-1 (NC235; NY-ESO-1 157-170, NW681; NY-ESO-1 143-154) or control HIV peptide-pulsed or NY-ESO-1 or SSX-2 protein-pulsed DC as APC. (B) Avidity of induced NY-ESO-1-specific Th1 cells was analyzed by ELISPOT assay using T-APC pulsed with serial dilutions of peptides. These experiments were performed independently at least twice with similar results. Data are expressed as mean±SD.

FIG. 3. S. typhimurium-NY-ESO-1 activates high avidity NY-ESO-1-specific CD4⁺ T cell precursors from naïve T cell population.

(A) CD4⁺CD25⁻ T cells isolated from PBMC of NC235 or NW681 were further separated into CD45RA⁺ or CD45RO⁺ cells using magnetic beads as described in Materials and Methods. These T cells were cultured with APC pulsed with a NY-ESO-1 peptide or infected with S. typhimurium-NY-ESO-1. Fifteen to 20 days later, specific T cell elicitation and capacity of elicited NY-ESO-1-specific Th1 cells to recognize naturally processed NY-ESO-1 protein was analyzed by ELISPOT assay using NY-ESO-1 (NC235; NY-ESO-1 157-170, NW681; NY-ESO-1 143-154) or control HIV peptide-pulsed or NY-ESO-1 or SSX-2 protein-pulsed DC as APC. (B) Avidity of induced NY-ESO-1-specific Th1 cells was analyzed by ELISPOT assay using T-APC pulsed with serial dilutions of peptides. These experiments were performed independently at least twice with similar results. Data are expressed as mean±SD.

FIG. 4. IL-6 and GITR signal is essential for elicitation of NY-ESO-1-specific CD4⁺ Th1 cells.

(A) Whole CD4⁺ T cells were isolated from PBMC of NC235 or NW681 and cultured with APC infected with S. typhimurium-NY-ESO-1 or S. typhimurium control strain without or with 10 μg/ml indicated antibodies. Fifteen to 20 days later, responses were analyzed by specific IFN-γ secretion for recognition of T-APC pulsed with NY-ESO-1 (NC235; NY-ESO-1 157-170, NW681; NY-ESO-1 143-154) or control HIV peptide. (B) Whole CD4⁺ T cells were isolated from PBMC of NC235 or NW681 and cultured with APC infected with S. typhimurium-NY-ESO-1 or pulsed with indicated NY-ESO-1 peptides with as well as recombinant IL-6 (10 ng/ml), GITRL-Fc (5 μg/ml), or both. Fifteen to 20 days later, responses were analyzed by specific IFN-γ secretion for recognition of T-APC pulsed with NY-ESO-1 (NC235; NY-ESO-1 157-170, NW681; NY-ESO-1 143-154) or control HIV peptide. (C) APC used for presensitization were prepared from PBMC of NC235 or NW681 and infected with S. typhimurium-NY-ESO-1. The kinetics of GITRL expression after S. typhimurium infection was analyzed. These experiments were performed independently at least twice with similar results. Data in (A) and (B) are expressed as mean±SD. * p<0.01, **p<0.05.

FIG. 5. S. typhimurium-induced CD4⁺ Th1 cells are resistant against suppression by CD4+CD25⁺ Tregs.

(A) CD4⁺CD25⁻ T cells isolated from PBMC of NC235 were cultured with APC pulsed with a NY-ESO-1 peptide or infected with S. typhimurium-NY-ESO-1 for 20 days. The presensitized T cells were labeled with CFSE as described in Materials and Methods and restimulated with T cell-depleted PBMC pulsed with NY-ESO-1 or control peptides for 6 days. The cells with diluted CFSE staining were gated and surface activation markers were analyzed as indicated. (B). CD4⁺CD25⁺ Tregs were isolated from PBMC and preactivated as described in Materials and Methods. S. typhimurium-induced or peptide-induced CD4+ Th1 cells were cultured with T cell-depleted PBMC pulsed with cognate NY-ESO-1 peptides in the absence or presence of these preactivated CD4⁺CD25⁺ Tregs and/or 10 μg/ml blocking anti-GITRL antibody. Proliferation was assessed as described in Materials and Methods. These experiments were performed independently at least twice with similar results. Data in (B) are expressed as mean±SD. * p<0.01.

FIG. 6. NY-ESO-1-specific CD4⁺ T cells are elicited by S. typhimurium-NY-ESO-1 from PBMC without CD4⁺CD25⁺ T cell depletion.

The presence or absence of NY-ESO-1 peptide-specific CD4⁺ Th1 cells in whole CD4⁺ T cells and CD4⁺ CD25⁻ T cells was analyzed by proliferation assays following a 15 to 20 day culture with APC pulsed with indicated NY-ESO-1 peptides (except NC155; NY-ESO-1 157-170) or infected with S. typhimurium-NY-ESO-1 or S. typhimurium control strain (see Materials and Methods). Responses were analyzed by specific ³H incorporation for recognition of autologous T cell-depleted PBMC pulsed with an indicated peptide (except NC155; NY-ESO-1 157-170 (grey column), or HIV peptide (open column)), in 4 healthy donors (A) and in 4 patients (B) with NY-ESO-1 expressing tumors but without NY-ESO-1 antibody. These experiments were performed independently at least twice with similar results. Data are expressed as mean±SD.

### DESCRIPTION OF SEQUENCES

SEQ ID NO: 1—Amino acid sequence of synthetic peptide of NY-ESO-1₈₇₋₉₈.

SEQ ID NO:2—Amino acid sequence of synthetic peptide of NY-ESO-1₁₂₁₋₁₃₂.

SEQ ID NO:3—Amino acid sequence of synthetic peptide of NY-ESO-1₁₄₃₋₁₅₄.

SEQ ID NO:4—Amino acid sequence of synthetic peptide of NY-ESO-1₁₅₇₋₁₇₀.

SEQ ID NO:5—Amino acid sequence of synthetic peptide of HIV P17₃₇₋₅₁.

### DETAILED DESCRIPTION OF THE INVENTION

Naturally occurring CD4+CD25+ regulatory T cells (Tregs) play an essential role in maintaining immunological balance and preventing the development of autoimmunity (Sakaguchi S et al., J. Immunol., 1995; 155:1151-1164; Maloy K J et al., Nat. Immunol., 2001; 2:816-822; Shevach E M, Nat Rev Immunol., 2002; 2:389-400; Kanamaru F et al., J. Immunol., 2004; 172:7306-7314) Accumulating evidence shows that Treg populations are also crucial for controlling anti-tumor immune responses. In mice, depletion of Treg populations enhances spontaneous and vaccine-induced anti-tumor T cell responses (Stephens G L et al., J. Immunol., 2004; 173:5008-5020; Onizuka S et al., Cancer Res., 1999; 59:3128-3133; Shimizu J et al., J. Immunol., 1999; 163:5211-5218; Steitz J et al., Cancer Res., 2001; 61:8643-8646; Sutmuller R P et al., J Exp Med., 2001; 194:823-832) and stimulation of CD4+CD25+ Tregs by immunization with self-antigens enhances the development of chemically-induced primary tumors (Nishikawa H et al., Proc Natl Acad Sci USA., 2003; 100:10902-10906) and of pulmonary metastases following injection of transplantable tumor cells (Nishikawa H et al., Proc Natl Acad Sci USA., 2005; 102:9253-9257). In humans, the presence of high numbers of CD4+CD25+ Tregs or high ratio of CD4+CD25+ Tregs to CD8+ T cells at the local tumor site is correlated with unfavorable prognosis (Curiel T J et al., Nat. Med., 2004; 10:942-949; Sato E et al., Proc Natl Acad Sci USA., 2005; 102:18538-18543). From these results, it is becoming an important priority to find strategies for controlling Tregs in the cancer vaccine field.

NY-ESO-1, a germ cell protein, was originally found by SEREX (serological identification of antigens by recombinant expression cloning) using the serum of an esophageal cancer patient (Sahin U et al., Proc Natl Acad Sci USA., 1995; 92:11810-11813 Chen Y T et al., Proc Natl Acad Sci USA., 1997; 94:1914-1918; Gnjatic S et al., Adv Cancer Res., 2006; 95:1-30). Its expression pattern and the frequent finding of humoral and cellular immune responses against this antigen in cancer patients with NY-ESO-1 expressing tumors make NY-ESO-1 one of the most intriguing cancer vaccine targets (Chen Y T et al., Proc Natl Acad Sci USA., 1997; 94:1914-1918; Gnjatic S et al., Adv Cancer Res., 2006; 95:1-30; Jäger E et al., J Exp Med., 1998; 187:265-270; Jager E et al., Proc Natl Acad Sci USA., 2000; 97:4760-4765). In monitoring a large series of cancer patients, humoral responses to NY-ESO-1 were found to be correlated with the presence of peripheral CD8+ T cells against NY-ESO-1, suggesting the involvement of CD4+ helper T cells in coordinating these responses. (Gnjatic S et al., Adv Cancer Res., 2006; 95:1-30; Jäger E et al., J Exp Med., 1998; 187:265-270; Jager E et al., Proc Natl Acad Sci USA., 2000; 97:4760-4765; Gnjatic S et al., Proc Natl Acad Sci USA., 2003; 100:8862-8867). It was indeed confirmed that effector CD4+ helper T cell responses to NY-ESO-1 were only observed in cancer patients who had antibodies against NY-ESO-1 (Gnjatic S et al., Adv Cancer Res., 2006; 95:1-30; Jager E et al., J Exp Med., 1998; 187:265-270; Jager E et al., Proc Natl Acad Sci USA., 2000; 97:4760-4765; Gnjatic S et al., Proc Natl Acad Sci USA., 2003; 100:8862-8867). However, it has recently been shown that NY-ESO-1-specific CD4+ T cell precursors are also present in patients with NY-ESO-1 expressing tumors but without NY-ESO-1 specific antibody as well as in healthy individuals and that CD4+CD25+ Tregs play a critical role in keeping these NY-ESO-1-specific precursors under control (Gnjatic S et al., Adv Cancer Res., 2006; 95:1-30; Nishikawa H et al., J. Immunol., 2006; 176:6340-6346; Nishikawa H et al., Blood., 2005; 106:1008-1011; Nishikawa H et al., J. Immunol., 2006; 176:6340-6346). The preexisting NY-ESO-1-specific CD4+ T cell precursors of seronegative and healthy individuals are exclusively from a naïve (CD4+CD25-CD45RA+) repertoire with high-avidity to antigen and are highly sensitive to Tregs, while spontaneously-induced NY-ESO-1-specific CD4+ T cells of seropositive patients are derived from an effector/memory (CD4+CD25-CD45RO+) repertoire with high-avidity to antigen but low sensitivity to Tregs (Gnjatic S et al., Adv Cancer Res., 2006; 95:1-30; Danke N A et al., J. Immunol., 2004; 172:5967-5972; Nishikawa H et al., Blood., 2005; 106:1008-1011). Vaccinating epithelial ovarian cancer patients with HLA-DPB1*0401/0402-restricted NY-ESO-1157-170 peptide in the presence of incomplete Freund's adjuvant results in the induction of NY-ESO-1-specific CD4+ T cells derived from an effector/memory (CD4+CD25-CD45RO+) repertoire with only low-avidity to antigen and low sensitivity to Tregs (Gnjatic S et al., Adv Cancer Res., 2006; 95:1-30; Nishikawa H et al., J Immunol., 2006; 176:6340-6346). These peptide vaccine-induced NY-ESO-1-specific T cells do not recognize naturally processed NY-ESO-1, while high-avidity naïve NY-ESO-1-specific T cell precursors are still present in peripheral blood but subject to continuous CD4+CD25+ Treg suppression throughout vaccination (Gnjatic S et al., Adv Cancer Res., 2006; 95:1-30; Nishikawa H et al., J Immunol., 2006; 176:6340-6346). Thus, a strategy to break ongoing suppression on preexisting high-avidity naïve T cell precursors is a critical element for an effective vaccine.

Recently, potential strategies including recombinant interleukin (IL)-2 diphtheria toxin conjugate DAB389IL-2 (targeting CD25, known as denileukin diftitax or ONTAK) and chemotherapy were exploited for eliminating CD4+CD25+ Treg populations in cancer vaccine therapies (Attia P et al., J Immunother., 2005; 28:582-592; Beyer M et al., Blood., 2005; 106:2018-2025; Dannull J et al., J Clin Invest., 2005; 115:3623-3633; Lutsiak M E et al., Blood., 2005; 105:2862-2868). Although the effect of Treg depletion remains to be determined in cancer patients, a recent report showed an enhancement of vaccine-mediated anti-tumor immunity (Dannull J et al., J Clin Invest., 2005; 115:3623-3633). However, an inherent risk to such systemic approaches is the emergence of potential autoimmunity. Glucocorticoid-induced TNF receptor (GITR) was originally reported as a molecule with direct functional relevance with regard to CD4+CD25+ Tregs, and stimulation of GITR on Tregs abrogates their suppressive activity (Shimizu J et al., Nat. Immunol., 2002; 3:135-142). A recent GITR knockout mouse study has revealed that the reversal of Treg suppression by GITR signaling is attributable to the costimulatory activity of agonistic anti-GITR antibody on the responder CD4+CD25⁻ T cells rather than a direct effect on Tregs (Stephens G L et al., J Immunol., 2004; 173:5008-5020; Shevach E M et al., Nat Rev Immunol., 2006; 6:613-618).

Recently, a number of studies shed new light on recognition of pathogen-associated molecular patterns through toll like receptors (TLRs) to break the suppressive environment present at the tumor local site (Iwasaki A et al., Nat. Immunol., 2004; 5:987-995; Pasare C et al., Science., 2003; 299:1033-1036; Pasare C et al., Immunity., 2004; 21:733-741; Yang Y et al., Nat. Immunol., 2004; 5:508-515; Peng G et al., Science., 2005; 309:1380-1384; Sutmuller R P et al., J Clin Invest., 2006; 116:485-494). TLR signals are not only able to block the suppressive activity of CD4+CD25+ Tregs but also break CD8 tolerance even in the presence of CD4+CD25+ Tregs (Iwasaki A et al., Nat. Immunol., 2004; 5:987-995; Pasare C et al., Science., 2003; 299:1033-1036; Pasare C et al., Immunity., 2004; 21:733-741; Yang Y et al., Nat. Immunol., 2004; 5:508-515; Peng G et al., Science., 2005; 309:1380-1384). Based on this evidence, viral and bacterial vectors that are able to stimulate TLR signaling are attracting much attention. We have recently reported that an avirulent recombinant strain of S. typhimurium endowed with the capacity to deliver NY-ESO-1 (S. typhimurium-NY-ESO-1) through its type III secretion system efficiently elicits NY-ESO-1-specific CD8+ and CD4+ T cell responses in vitro (when depleted of CD4+CD25+ Tregs) from blood of cancer patients with spontaneous immunity, while immunization with this construct in mice results in the regression of pre-established NY-ESO-1 expressing tumors (Russmann H et al., Science., 1998; 281:565-568; Nishikawa H et al., J Clin Invest., 2006; 116:1946-1954).

Certain aspect described herein address the capacity of S. typhimurium to overcome the suppressive activity of CD4+CD25+ Tregs. In the presence of CD4+CD25+ Tregs, S. typhimurium was able to elicit high-avidity NY-ESO-1-specific CD4+ T cell responses from naïve T cell precursors derived from healthy individuals and patients with NY-ESO-1 expressing tumors but seronegative for NY-ESO-1. IL-6 and GITR signaling synergistically worked to break the suppressive activity of CD4+CD25+ Tregs. Furthermore, S. typhimurium-induced CD4+ T helper type 1 (Th1) cells expressed GITRand were resistant to suppression by CD4+CD25+ Tregs, and this resistance was abrogated by blocking GITR signaling.

In certain aspects of the invention, methods for stimulating an immune response against an antigen are provided. Immune responses are well known to those of ordinary skill in the art, and it is contemplated that an immune response can be a cell mediated response and/or an antibody response. The immune responses stimulated in accordance with the invention include Th1 responses, Th2 responses, or mixed Th1/Th2 responses. Accordingly, the stimulated immune response can be a CD8+ T cell response, a CD4+ T cell response or both a CD8+ T cell response and a CD4+ T cell response. In some embodiments, stimulation of an immune response includes stimulation of a pre-existing immune response that existed through previous exposure to antigen.

An “antigen”, as used herein, is a substance that stimulates a specific immune response; for the purposes of this invention, antigens include both tumor antigens and non-tumor antigens. Antigens encoded and delivered by the avirulent recombinant bacteria of the invention are polypeptides.

Non-tumor antigens include without limitation virus antigens and antigens of a microorganism. Viral antigens include, but are not limited to, antigens of: influenza virus, Epstein-Barr virus and Varicella zoster virus. Viral antigens also include antigens of viruses that cause viral diseases, including: mumps, measles, rubella, chicken pox, influenza, hepatitis A, hepatitis B and polio. Antigens of a microorganism include, but are not limited to, antigens of microorganisms that cause diphtheria, tetanus, pertussis, bacterial meningitis (Haemophilus influenza type B), and Streptococcus pneumoniae antigen (invasive pneumococcal disease).

A tumor antigen as used herein refers to tumor antigen polypeptides, which includes tumor antigen proteins or fragments thereof, and tumor antigen peptides. Preferred tumor antigens include, but are not limited to: NY-ESO-1, a MAGE antigen, for example MAGE-A3, a SSX antigen, for example SSX2, SCP1, CT7, NY-CO-58, a BAGE antigen, a GAGE antigen, Melan-A/MART-1, gp100 and gp75. Cancer-testis antigens, which are expressed only or primarily in cancer cells and testis cells, are preferred tumor antigens. A list of some cancer-testis antigens is provided in Table 1 (Cancer Immunity, 4:1 (2004) Table 1).

TABLE 1
A list of some CT genes: 44 CT gene families,
89 individual genes or isoforms

| | Transcript family | |
|---|---|---|
| | CT | |
| Transcript | Identifier | Family Members/CT Identifier |
| MAGEA | CT1 | MAGEA1/CT1.1, MAGEA2/CT1.2, |
| | | MAGEA3/CT1.3, MAGEA4/CT1.4, |
| | | MAGEA5/CT1.5, MAGEA6/CT1.6, |
| | | MAGEA7/CT1.7, MAGEA8/CT1.8, |
| | | MAGEA9/CT.9, MAGEA10/CT1.10, |
| | | MAGEA11/CT1.11, |
| | | MAGEA12/CT1.12 |
| BAGE | CT2 | BAGE/CT2.1, BAGE2/CT2.2, |
| | | BAGE3/CT2.3, BAGE4/CT2.4, |
| | | BAGE5/CT2.5 |
| MAGEB | CT3 | MAGEB1/CT3.1, MAGEB2/CT3.2, |
| | | MAGEB5/CT3.3, MAGEB6/CT3.4 |
| GAGE1 | CT4 | GAGE1/CT4.1, GAGE2/CT4.2, |
| | | GAGE3/CT4.3, GAGE4/CT4.4, |
| | | GAGE5/CT4.5, GAGE6/CT4.6, |
| | | GAGE7/CT4.7, GAGE8/CT4.8 |
| SSX | CT5 | SSX1/CT5.1, SSX2/CT5.2a, |
| | | SSX2/CT5.2b, SSX3/CT5.3, |
| | | SSX4/CT5.4 |
| NY-ESO-1 | CT6 | NY-ESO-1/CT6.1, LAGE-1a/CT6.2a, |
| | | LAGE-1b/CT6.2b |
| MAGEC1 | CT7 | MAGEC1/CT7.1, MAGEC3/CT7.2 |
| SYCP1 | CT8 | SYCP1/CT8 |
| BRDT | CT9 | BRDT/CT9 |
| MAGEE1 | CT10 | MAGEE1/CT10 |
| CTp11/SPANX | CT11 | SPANXA1/CT11.1, |
| | | SPANXB1/CT11.2, SPANXC/CT11.3, |
| | | SPANXD/CT11.4 |
| XAGE- | CT12 | XAGE-1a/CT12.1a, XAGE- |
| 1/GAGED | | 1b/CT12.1b, XAGE-1c/CT12.1c, |
| | | XAGE-1d/CT12.1d, XAGE-2/CT12.2, |
| | | XAGE-3a/CT12.3a, XAGE- |
| | | 3b/CT12.3b, XAGE-4/CT12.4 |
| HAGE | CT13 | HAGE/CT13 |
| SAGE | CT14 | SAGE/CT14 |
| ADAM2 | CT15 | ADAM2/CT15 |
| PAGE-5 | CT16 | PAGE-5/CT16.1, CT16.2 |
| LIP1 | CT17 | LIP1/CT17 |
| NA88 | CT18 | NA88/CT12 |
| IL13RA1 | CT19 | IL13RA1/CT19 |
| TSP50 | CT20 | TSP50/CT20 |
| CTAGE-1 | CT21 | CTAGE-1/CT21.1, CTAGE-2/CT21.2 |
| SPA17 | CT22 | SPA17/CT22 |
| OY-TES-1 | CT23 | OY-TES-1/CT23 |
| CSAGE | CT24 | CSAGE/CT24.1, TRAG3/CT24.2 |
| MMA1/DSCR8 | CT25 | MMA-1a/CT25.1a, MMA-1b/CT25.1b |
| CAGE | CT26 | CAGE/CT26 |
| BORIS | CT27 | BORIS/CT27 |
| HOM-TES-85 | CT28 | HOM-TES-85/CT28 |
| AF15q14/D40 | CT29 | D40/CT29 |
| E2F- | CT30 | HCA661/CT30 |
| like/HCA661 | | |
| PLU-1 | CT31 | PLU-1/CT31 |
| LDHC | CT32 | LDHC/CT32 |
| MORC | CT33 | MORC/CT33 |
| SGY-1 | CT34 | SGY-1/CT34 |
| SPO11 | CT35 | SPO11/CT35 |
| TPX1 | CT36 | TPX-1/CT36 |
| NY-SAR-35 | CT37 | NY-SAR-35/CT37 |
| FTHL17 | CT38 | FTHL17/CT38 |
| NXF2 | CT39 | NXF2/CT39 |
| TAF7L | CT40 | TAF7L/CT40 |
| TDRD1 | CT41 | TDRD1/CT41.1, NY-CO-45/CT41.2 |
| TEX15 | CT42 | TEX15/CT42 |
| FATE | CT43 | FATE/CT43 |
| TPTE | CT44 | TPTE/CT44 |

Other aspects of the invention provide methods for expressing a polypeptide as described herein, using avirulent bacteria. In exemplary embodiments the avirulent bacteria include a type III secretion system. Such bacteria are known to those of ordinary skill in the art. Briefly, such bacteria provide delivery of proteins to a cell cytosol without the requirement of the bacteria entering the target cell. Further details can be found in Russmann, H. et al., Science, 281:565-8, 1998; Stebbins, C. E. et al., Nature, 414:77-81, 2001. Bacteria with Type III secretion systems include but are not limited to Salmonella spp., Yersinia spp., Bordetella spp., Escherichia coli, Shigella spp., Burkholderia mallei, Burkholderia pseudomallei, and Pseudomonas aeruginosa. In a preferred embodiment, an avirulent bacteria is Salmonella typhimurium. In particularly preferred embodiment, an avirulent bacteria is ΔphoP-phoQ Salmonella typhimurium. In a specific embodiment, an avirulent bacteria is Salmonella typhimurium pSB2470, described in Russmann et al. 1998. Science. 281:565-568.

A virulent bacteria are grown and maintained in sterile media under bacterial growth conditions as known to those of ordinary skill in the art. In some instances the avirulent bacteria may contain a deletion in the asd gene encoding aspartate semialdehyde dehydrogenase involved in the synthesis of diaminopimelic acid (DAP), an essential component of the peptidoglycan layer of the bacterial envelope. Bacteria lacking the asd gene can only grow in medium containing DAP. It is contemplated that the avirulent bacteria in these instances are grown in the presence of diaminopimelic acid (DAP) to aid replication. Mammals do not produce DAP therefore bacteria lacking the asd gene are able to replicate for a limited number of generations, rendering the bacteria avirulent. The asd gene deletion removes the need for using antibiotics. The removal of the need for antibiotics ensures the vaccine complies with FDA regulations.

As used herein, a “nucleic acid molecules encoding” means the nucleic acid molecules that code for the antigen polypeptides or immunogenic fragments thereof. These nucleic acid molecules may be DNA or may be RNA (e.g. mRNA). The nucleic acid molecules encoding (tumor) antigen(s) also encompass variants of the nucleic acid molecules described herein. These variants may be splice variants or allelic variants of certain antigen-encoding sequences. Variants of the nucleic acid molecules of the invention are intended to include homologs and alleles which are described further below. Further, as used herein, the term “tumor antigen molecules” includes tumor antigens (polypeptides and fragments thereof) as well as tumor antigen nucleic acids. In all embodiments, human tumor antigens and the encoding nucleic acid molecules thereof are preferred.

In one aspect, the invention provides isolated nucleic acid molecules that encode the antigens for use in the methods and compositions described herein. As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art.

The nucleic acid molecules of the invention that encode antigen(s) are also intended to encompass homologs and alleles which can be identified by conventional techniques as well as by in silico (i.e., computer-based homology screening using computer software). Identification of human homologs and homologs of other organisms (i.e., orthologs) of tumor antigen polypeptides will be familiar to those of skill in the art. In general, nucleic acid hybridization is a suitable method for identification of homologous sequences of another species which correspond to a known sequence. Standard nucleic acid hybridization procedures can be used to identify related nucleic acid sequences of selected percent identity. For example, one can construct a library of cDNAs reverse transcribed from the mRNA of a selected tissue and use the nucleic acids that encode selected tumor antigen(s), such as those identified herein, to screen the library for related nucleotide sequences. The screening preferably is performed using high-stringency conditions to identify those sequences that are closely related by sequence identity. Nucleic acids so identified can be translated into polypeptides and the polypeptides can be tested for activity.

The term “high stringency” as used herein refers to parameters with which the art is familiar. Nucleic acid hybridization parameters may be found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C. The temperature of the wash may be adjusted to provide different levels of stringency. For example the wash can be performed at temperatures of 42° C., 45° C., 50° C., 55° C., 60° C., 65° C. or 68° C. The skilled artisan would be able to adjust the conditions to determine the optimum conditions as required.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of the tumor antigen nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

Optimal alignment of sequences for comparison may alternatively be conducted using programs such as BLAST, publicly available on the National Library of Medicine website. Other programs such as UniGene (The National Library of Medicine website), SAGE Anatomic Reviewer and its Virtual Northern tool, (The Cancer Genome Anatomy Project CGAP website) are also publicly available. One of ordinary skill in the art would be able to use these programs to align sequences and determine percentage identity with no more than routine experimentation.

In general, homologs and alleles typically will share at least 90% nucleotide identity and/or at least 95% amino acid identity to the sequences of cancer-testis nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In another aspect of the invention, fragments are contemplated. A fragment includes but is not limited to a fragment of a nucleic acid or a fragment of a polypeptide molecule. In an embodiment a fragment is an immunogenic fragment. For polypeptides, the fragment can be at least about 5, 6, 7, 8, 9, 10, 11, 12, 13, 14, 15, 16, 17, 18, 19, 20, 25, 30, 35, 40, 45, 50, 75, or 100 amino acids in length. For nucleic acid molecules the fragment can be at least about 12, 13, 14, 15, 16, 17, 18, 19, 20, 21, 22, 23, 24, 25, 26, 27, 28, 29, 30, 31 and 32 bases long. In some embodiments a nucleic acid fragment can be at least about 35, 40, 45, 50, 55, 60, 65, 70, 75, 100, 150, 200, 250, 300, or 500 nucleotides in length.

In an exemplary embodiment of the invention the polypeptides are fusion proteins. In an embodiment the fusion protein consists of a tumor antigen polypeptide (e.g., NY-ESO-1) or an immunogenic fragment thereof fused to a polypeptide that is secreted by a type III secretion system. Examples of such peptides include: SptP, SipA, SipB, SipC, SipD, InvJ, SpaO, AvrA, and SopE proteins of Salmonella, the Yop and Ypk proteins of Yersinia (for example, YopE, YopH, YopM and YpkA), the Ipa proteins of Shigella, and the ExoS proteins of Pseudomonas aeruginosa.

Fragments of the immunogenic tumor antigen polypeptides (including immunogenic peptides) also can be synthesized chemically using well-established methods of peptide synthesis. Thus, fragments of the disclosed polypeptides are useful for eliciting an immune response and for assaying for the presence of antibodies, or other similar molecules such as T cell receptors. In one embodiment fragments of a polypeptide which comprises any of SEQ ID NO: 1-5 or fragments thereof that are at least eight amino acids in length and exhibit immunogenicity are provided. The fragments may be any length from 8 amino acids up to one amino acid less than the full length size of polypeptide. Specific embodiments provide fragments of a polypeptide which comprise the polypeptide sequences set forth as any of SEQ ID NO: 1-5 or the fragments described above.

Fragments of a polypeptide preferably are those fragments that retain a distinct functional capability of the polypeptide. Functional capabilities that can be retained in a fragment of a polypeptide include interaction with antibodies or MHC molecules (e.g. immunogenic fragments), interaction with other polypeptides or fragments thereof, selective binding of nucleic acids or proteins, and enzymatic activity. One important activity is the ability to provoke in a subject an immune response. As will be recognized by those skilled in the art, the size of the fragment that can be used for inducing an immune response will depend upon factors such as whether the epitope recognized by an antibody is a linear epitope or a conformational epitope or the particular MHC molecule that binds to and presents the fragment (e.g. HLA class I or II). Thus, some immunogenic fragments of tumor antigen polypeptides will consist of longer segments while others will consist of shorter segments, (e.g. about 8, 9, 10, 11, 12, 13, 14, 15, 16 or more amino acids long, including each integer up to the full length of the polypeptide). Those skilled in the art are well versed in methods for selecting immunogenic fragments of polypeptides.

The invention embraces variants of the tumor antigen polypeptides described above. As used herein, a “variant” of a tumor antigen polypeptide is a polypeptide which contains one or more modifications to the primary amino acid sequence of a tumor antigen polypeptide. Modifications which create a tumor antigen variant can be made to a tumor antigen polypeptide 1) to reduce or eliminate an activity of a tumor antigen polypeptide; 2) to enhance a property of a tumor antigen polypeptide, such as protein stability in an expression system or the stability of protein-protein binding; 3) to provide a novel activity or property to a tumor antigen polypeptide, such as addition of an antigenic epitope or addition of a detectable moiety; or 4) to provide equivalent or better binding to a MHC molecule.

Modifications to a tumor antigen polypeptide are typically made to the nucleic acid which encodes the tumor antigen polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the tumor antigen amino acid sequence. One of skill in the art will be familiar with methods for predicting the effect on protein conformation of a change in protein sequence, and can thus “design” a variant tumor antigen polypeptide according to known methods. One example of such a method is described by Dahiyat and Mayo in Science 278:82-87, 1997, whereby proteins can be designed de novo. The method can be applied to a known protein to vary only a portion of the polypeptide sequence. By applying the computational methods of Dahiyat and Mayo, specific variants of a tumor antigen polypeptide can be proposed and tested to determine whether the variant retains a desired conformation.

In general, variants include tumor antigen polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a tumor antigen polypeptide by eliminating proteolysis by proteases in an expression system (e.g., dibasic amino acid residues in yeast expression systems in which KEX2 protease activity is present).

Mutations of a nucleic acid which encode a tumor antigen polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant tumor antigen polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a tumor antigen gene or cDNA clone to enhance expression of the polypeptide. The activity of variants of tumor antigen polypeptides can be tested by cloning the gene encoding the variant tumor antigen polypeptide into a bacterial or mammalian expression vector, introducing the vector into an appropriate host cell, expressing the variant tumor antigen polypeptide, and testing for a functional capability of the tumor antigen polypeptides as disclosed herein. For example, the variant tumor antigen polypeptide can be tested for reaction with autologous or allogeneic sera. Preparation of other variant polypeptides may favor testing of other activities, as will be known to one of ordinary skill in the art.

The skilled artisan will also realize that conservative amino acid substitutions may be made in immunogenic tumor antigen polypeptides to provide functionally equivalent variants, or homologs of the foregoing polypeptides, i.e., the variants retain the functional capabilities of the immunogenic tumor antigen polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the tumor antigen polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the tumor antigen disclosed herein and retain the specific antibody-binding characteristics of the antigens.

Likewise, upon determining that a peptide derived from a tumor antigen polypeptide is presented by an MHC molecule and recognized by antibodies or T lymphocytes (e.g., helper T cells or CTLs), one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or T lymphocytes when bound to MHC. These variants can be tested for improved stability and are useful, inter alia, in vaccine compositions.

Conservative amino-acid substitutions in the amino acid sequence of tumor antigen polypeptides to produce functionally equivalent variants of tumor antigen polypeptides typically are made by alteration of a nucleic acid encoding a tumor antigen polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a tumor antigen polypeptide. Where amino acid substitutions are made to a small unique fragment of a tumor antigen polypeptide, such as an antigenic epitope recognized by autologous or allogeneic sera or T lymphocytes, the substitutions can be made by directly synthesizing the peptide. The activity of functionally equivalent variants of tumor antigen polypeptides can be tested by cloning the gene encoding the altered tumor antigen polypeptide into a bacterial or mammalian expression vector, introducing the vector into an appropriate host cell, expressing the altered polypeptide, and testing for a functional capability of the tumor antigen polypeptides as disclosed herein. Peptides that are chemically synthesized can be tested directly for function, e.g., for binding to antisera recognizing associated antigens.

Methods for preparing antigen presenting cells and methods for preparing antigen-specific T cells from peripheral blood mononuclear cells also are provided. Peripheral blood mononuclear cells (PBMCs) are obtained from a subject using methods known to those of skill in the art. The PBMCs are infected with a recombinant avirulent bacteria having a type III secretion system that expresses an antigen. The PBMCs are cultured using media and methods described in the examples and known to those of ordinary skill in the art. The PBMCs expressing the antigen are isolated as antigen presenting cells (APCs). Antigen-specific T cells are prepared by contacting T-cells, which are isolated from PBMCs, with APCs. In a preferred embodiment, antigen-specific T cells are prepared by contacting CD4⁺ T cells, which are not depleted of CD4+CD25+ T cells (i.e., Tregs), with APCs. Cell isolation techniques are known to those of ordinary skill in the art and include cell sorting (e.g. FACS), immunoprecipitation, centrifugation etc.

According to the invention, the terms “treating” and “treatment” include prophylaxis and therapy. When provided prophylactically, a treatment may be administered to a subject in advance of cancer (e.g., to a subject at risk of cancer) or upon the development of early signs of cancer in a subject. A prophylactic treatment serves to prevent, delay, or reduce the rate of onset of cancer or the appearance of symptoms associated with cancer. When provided therapeutically, a treatment may be administered at (or after) the onset of the appearance of symptoms of actual cancer. Therapy includes preventing, slowing, stopping, or reversing cancer or certain symptoms associated with cancer. In some embodiments, a treatment may serve to reduce the severity and duration of cancer or symptoms thereof. In some embodiments, treating a subject may involve halting or slowing the progression of cancer or of one or more symptoms associated with cancer. In some embodiments, treating a subject may involve preventing, delaying, or slowing the onset or progression of long-term symptoms associated with cancer. In some embodiments, treating a subject may involve complete or partial remission.

As used herein, a “subject” is a mammal, preferably a human, non-human primate, cow, horse, pig, sheep, goat, dog, cat or rodent. In all embodiments, human subjects are preferred. A “subject in need” and “subject in need of treatment” as used herein is a subject that is suspected of having cancer or has been diagnosed with cancer. A subject in need is also a subject at risk of having cancer as determined by associated risk factors including but not limited to smoking, family history, genetic predisposition, and external factors (for example environmental factors).

For human cancers, particular examples include, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma, neurosarcoma, chondrosarcoma, Ewing sarcoma, malignant fibrous histocytoma, glioma, esophageal cancer, hepatoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; testicular cancer; thyroid cancer including thyroid adenocarcinoma and medullar carcinoma; transitional cancer and renal cancer including adenocarcinoma and Wilms tumor.

The invention also contemplates the use of the methods of the invention in combination with conventional cancer treatment methods and procedures. Conventional treatment for cancer may include, but is not limited to: surgical intervention, chemotherapy, radiotherapy, and adjuvant systemic therapies. In one aspect of the invention, treatment may include administering binding polypeptides such as antibodies that specifically bind to the tumor antigen. These binding polypeptides can be optionally linked to one or more detectable markers, antitumor agents or immunomodulators.

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models, can be used for testing of immunization against cancer.

Methods for immunization, including formulation of a vaccine composition and selection of doses, route of administration and the schedule of administration (e.g. primary and one or more booster doses), are well known in the art. The tests also can be performed in humans, where the end point can be to test for the presence of enhanced levels of circulating T-cells against cells bearing the antigen, to test for levels of circulating antibodies against the antigen, to test for the presence of cells expressing the antigen and so forth. It is further contemplated that immunization may be performed using a kit which includes a DNA vaccine vector encoding an antigen and a recombinant avirulent bacteria having a type III secretion system that expresses the antigen. This combination could be seen as a booster for a vaccine that a patient has already received, plus the recombinant bacteria. Alternatively, for tumor antigens, it can be used essentially as a prime-boost combination product. The kit may contain more than one dose of a vaccine against an antigen.

As part of the immunization compositions, recombinant bacteria expressing via a type III secretion system one or more tumor antigen polypeptides or immunogenic fragments thereof are administered, optionally with one or more adjuvants, to induce an immune response or to increase an immune response. In some embodiments, immunization is achieved via administration of a bacteria having a type III secretion system, which does not express one or more tumor antigen polypeptides or immunogenic fragments thereof, and one or more soluble tumor antigen polypeptides, or immunogenic fragments thereof, optionally with one or more adjuvants, to induce an immune response or to increase an immune response. In some embodiments, immunization is achieved via administration of one or more soluble tumor antigen polypeptides or immunogenic fragments thereof, optionally with one or more adjuvants, and optionally with a bacteria having a type III secretion system, to induce an immune response or to increase an immune response. In some embodiments, immunization is achieved via administration of one or more soluble tumor antigen polypeptides, or immunogenic fragments thereof, with a Glucoricoid-induced receptor agonist (e.g., GITRL, GITRL-Fc), optionally with IL-6, one or more adjuvants, and/or a bacteria having a type III secretion system, to induce an immune response or to increase an immune response. Immunization can also be achieved passively by administering a population of antigen presenting cells (APCs) and/or T cells to a subject, wherein the T cells recognize one or more antigens.

An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham), ISCOM (CSL Ltd., Parkville, Victoria, Australia) derived from the bark of the Quillaia saponaria molina tree; QS-7, QS-17, QS-18, and QS-L1 (So et al., Mol. Cells. 7:178-186, 1997); incomplete Freund's adjuvant; complete Freund's adjuvant; montanide; alum; CpG oligonucleotides (see e.g. Kreig et al., Nature 374:546-9, 1995) and other immunostimulatory oligonucleotides; various water-in-oil emulsions prepared from biodegradable oils such as squalene and/or tocopherol; and factors that are taken up by the so-called ‘toll-like receptor 7’ on certain immune cells that are found in the outside part of the skin, such as imiquimod (3M, St. Paul, Minn.). Preferably, the antigens are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of polypeptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens. There are a number of additional immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation, and effector function. B7 also interacts with CTLA4 (CD152) on T cells and studies involving CTLA4 and B7 ligands indicate that the B7-CTLA4 interaction can enhance antitumor immunity and CTL proliferation (Zheng et al., Proc. Nat'l Acad. Sci. USA 95:6284-6289, 1998).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol. 154:5637-5648, 1995). Tumor cell transfection with B7 has been discussed in relation to in vitro CTL expansion for adoptive transfer immunotherapy by Wang et al. (J. Immunother. 19:1-8, 1996). Other delivery mechanisms for the B7 molecule would include nucleic acid (naked DNA) immunization (Kim et al., Nature Biotechnol. 15:7:641-646, 1997) and recombinant viruses such as adeno and pox (Wendtner et al., Gene Ther. 4:726-735, 1997). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642, 1997; Fenton et al., J. Immunother., 21:95-108, 1998).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J Immunother., 21(2):95-108, 1998). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature 393:474, 1998; Bennett et al., Nature 393:478, 1998; Schoenberger et al., Nature 393:480, 1998). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor associated antigens which are normally encountered outside of an inflammatory context or are presented by non-professional APCs (tumor cells). Other methods for inducing maturation of dendritic cells, e.g., by increasing CD40-CD40L interaction, or by contacting DCs with CpG-containing oligodeoxynucleotides or stimulatory sugar moieties from extracellular matrix, are known in the art. In these situations Th help and B7 costimulation signals are not provided. This mechanism might be used in the context of antigen pulsed DC based therapies or in situations where Th epitopes have not been defined within known tumor associated antigen precursors.

Glucocorticoid-induced TNF receptor (GITR), also referred to as TNFRSF18 or AITR, is a member of the tumor necrosis factor (TNF) receptor family. As used herein, glucocorticoid-induced TNF receptor agonists embrace molecules that bind to and activate glucocorticoid-induced TNF receptor (GITR). Examples of glucocorticoid-induced TNF receptor agonists are known in the art (for example, Stone G W, et al., J. Virol. 2006 February; 80 (4):1762-72 and US Patent Application #20070098719). In one embodiment, the glucocorticoid-induced TNF receptor ligand (GITRL), or a functional fragment thereof, is a glucocorticoid-induced TNF receptor agonist. As used herein a GITRL fragment is a polypeptide derived from GITRL that binds to and activates Glucocorticoid-induced TNF receptor (GITR). Thus, a GITRL fragment is a polypeptide derived from GITRL that is a GITR agonist. In some embodiments a fusion protein comprising an Fc fragment fused to the glucocorticoid-induced TNF receptor ligand (GITRL-Fc), or a functional fragment thereof, is a glucocorticoid-induced TNF receptor agonist. In some embodiments, a glucocorticoid-induced TNF receptor agonist is an antibody (e.g., Catalog Number 14-5874, mAb against GITR, Clone DTA-1 eBiosciences, Inc.), or antibody fragment, that binds to and activates GITR.

Antibodies (e.g., GTIR agonists) include polyclonal and monoclonal antibodies, prepared according to conventional methodology. An antibody which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral (including nasal, vaginal, rectal, mucosal surface), intravenous, intratumoral, intraperitoneal, intramuscular, intracavity, subcutaneous, or transdermal. In preferred embodiments the composition is administered orally or intratumorally.

The preparations of the invention are administered in effective amounts. An effective amount is that amount of a pharmaceutical preparation that alone, or together with further doses, stimulates the desired response. In the case of treating cancer, the desired response is inhibiting the progression of the cancer. This may involve only slowing the progression of the disease temporarily, although more preferably, it involves halting the progression of the disease permanently. In the case of inducing an immune response, the desired response is an increase in antibodies or T lymphocytes which are specific for the tumor antigen(s) employed. These desired responses can be monitored by routine methods.

Where it is desired to stimulate an immune response using a therapeutic composition of the invention, this may involve the stimulation of a humoral antibody response resulting in an increase in antibody titer in serum, a clonal expansion of cytotoxic lymphocytes, or some other desirable immunologic response. It is believed that doses of immunogens ranging from one nanogram/kilogram to 100 milligrams/kilogram, depending upon the mode of administration, would be effective. The preferred range is believed to be between 500 nanograms and 500 micrograms per kilogram. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

It is further contemplated that administration of the therapeutic composition of the invention, when administered in combination with other factors can occur subsequently, concurrently, or simultaneously. One of ordinary skill in the art would be able to determine the required administration procedure using no more than routine experimentation.

### EXAMPLES

### Example 1

### Materials and Methods for the Induction of Regulatory T Cell-Resistant Helper CD4⁺ T Cells by Bacterial Vector

### Donor Samples

All healthy donors were subjects with no history of autoimmune disease. All patients had NY-ESO-1 expressing melanoma except NW1060 with sarcoma. The HLA typing of healthy donors and patients except NC235 who is HLA-DPB1*0401 was described previously (Nishikawa H et al., Blood., 2005; 106:1008-1011). All samples were collected after informed consent as a part of study approved by the Ethics Committee of Landesarztekammer Hessen, Frankfurt.

### Reagents

Synthetic peptides NY-ESO-1₈₇₋₉₈ (SEQ ID NO: 1—LLEFYLAMPFAT), NY-ESO-1₁₂₁₋₁₃₂ (SEQ ID NO: 2—VLLKEFTVSGNI), NY-ESO-1₁₄₃₋₁₅₄ (SEQ ID NO: 3—RQLQLSISSCLQ), NY-ESO-1₁₅₇₋₁₇₀ (SEQ ID NO: 4-SLLMWITQCFLPVF), and HIV P17₃₇₋₅₁ (SEQ ID NO: 5—ASRELERFAVNPGLL) were obtained from Bio-Synthesis (Lewisville, Tex.) (Nishikawa H et al., Blood., 2005; 106:1008-1011). Recombinant full-length NY-ESO-1 or SSX-2 proteins were prepared using procedures described previously (Stockert E et al., J Exp Med., 1998; 187:1349-1354). Anti-IL-4 (MP4-25D2, rat IgG1), anti-IL-6 (MQ2-13A5, rat IgG1), anti-IL-12 (C8.6, mouse IgG1), anti-IFN-gamma (NIB42, mouse IgG1), anti-TNF-alpha (MAb1, mouse IgG1) and phycoerythrin (PE)-conjugated anti-CD62L (DREG-56, mouse IgG1) antibodies were purchased from eBioscience (San Diego, Calif.). Blocking anti-GITRL (109101, mouse IgG1) monoclonal antibody and recombinant IL-6 were purchased from R&D Systems (Minneapolis, Minn.). PE-conjugated anti-GITRL (EB11-2, mouse IgG1) mAb was purchased from BioLegend (San Diego, Calif.). PE-conjugated anti-CD25 (M-A251, mouse IgG1), anti-CD44 (515, mouse IgG1), anti-CD154 (TRAP1, mouse IgG1) and anti-CD45RO (UCHL1, mouse IgG2a) antibodies were purchased from BD Biosciences (Franklin Lakes, N.J.). Purified anti-CD3 mAb (UCHT1, mouse IgG1) was purchased from BD Biosciences. A plasmid encoding human GITRL was purchased from Invitrogen (Carlsbad, Calif.). GITRL cDNA was subcloned into pFUSE-Fc, a mammalian expression vector for Fc-fusion protein (Invivogen, San Diego). This plasmid was transfected into COS-7 cells to produce recombinant GITRL-Fc protein in culture supernatant. Then, secreted GITRL-Fc protein was collected and purified with Protein A beads according to the manufacturer's protocol (PIERCE, Rockford, Ill.).

S. typhimurium Infection

The S. typhimurium AphoP-phoQ strain recombinant for NY-ESO-1 and control strains have been described previously (Russmann H et al., Science., 1998; 281:565-568; Nishikawa H et al., J Clin Invest., 2006; 116:1946-195). S. typhimurium was grown as described previously (Russmann H et al., Science., 1998; 281:565-568; Nishikawa H et al., J Clin Invest., 2006; 116:1946-195). Target cells were infected with the different S. typhimurium strains for 1 hour at 37° C. with at a multiplicity of infection (MOI) of 40. Extracellular bacteria were killed by transferring the target cells to RPMI 1640 medium containing 100 μg/ml gentamicin (Sigma) and incubating at 37° C. for 1 hour (Nishikawa H et al., J Clin Invest., 2006; 116:1946-195). Infected cells were subsequently used as APC.

### Generation of NY-ESO-1-Specific CD4⁺ T Cells

NY-ESO-1-specific CD4⁺ T cells were elicited as described previously (Nishikawa H et al., Blood., 2005; 106:1008-1011). Briefly, CD4⁺ T cells and CD4+CD25⁻ T cells were isolated from PBMC using CD4⁺CD25⁺Regulatory T Cell Isolation Kit (Miltenyi Biotec). In some experiments, CD4+CD25⁻ T cells were further separated into CD45RO-depleted T cells (CD4⁺CD25⁻CD45RA⁺ T cells) or CD45RA-depleted T cells (CD4⁺CD25⁻CD45RO⁺ T cells) using CD45RO Microbeads or CD45RA Microbeads (Miltenyi Biotec), respectively (Nishikawa H et al., Blood., 2005; 106:1008-1011). The purity of selected populations was confirmed to be >96% (Nishikawa H et al., Blood., 2005; 106:1008-1011). Non-CD4⁺ cells retained in the MACS separation column were flushed out and were used to prepare APC. These non-CD4⁺ cells were allowed to adhere to tissue culture plates (Corning Glass, Corning, N.Y.) for 2 hours, and non-adherent cells were removed by washing. The adherent cells pulsed with 10 μM of one or two peptides overnight or infected with S. typhimurium were used as APC. After irradiation, 5×10⁵ APC were added to round-bottom 96-well plates (Corning) containing 1-5×10⁵ unfractionated CD4⁺, CD4⁺CD25⁻, CD4⁺CD25⁻CD45RO⁺ or CD4⁺CD25⁻CD45RA⁺ T cells and were fed with 10 U/ml IL-2 (Roche Molecular Biochemicals, Indianapolis, Ind.). Subsequently, one-half of medium was replaced by fresh medium containing IL-2 (20 U/ml) twice per week.

### ELISPOT Assay

The number of IFN-γ secreting antigen-specific CD4⁺ T cells was assessed by ELISPOT assays as described previously (Gnjatic S et al., Proc Natl Acad Sci USA., 2003; 100:8862-8867; Nishikawa H et al., Blood., 2005; 106:1008-1011; Atanackovic D et al., J Immunol Methods., 2003; 278:57-66). Briefly, flat-bottomed, 96-well nitrocellulose-coated microtiter plates (Millipore, Bedford, Mass.) were coated with anti-IFN-γ antibody (1-D1K; MABTECH, Stockholm). The presensitized T cells and phytohaemagglutinin (PHA HA15; Murex Diagnostics, Dartford, U.K.) activated CD4+ T cells (Atanackovic D et al., J Immunol Methods., 2003; 278:57-66) or DC pulsed with 10 μM or indicated amount of peptides, or 25 μg/ml protein overnight were added to each well and incubated for 24 hours. Spots were developed using biotinylated anti-IFN-γ antibody (7-B6-1-biotin; MABTECH), alkaline phosphatase conjugated streptavidin (Roche Diagnostics GmbH, Mannheim, Germany) and 5-bromo-4-chloro-3-indolyl phosphate/nitroblue tetrazolium (Sigma) and counted with C. T. L. Immunospot analyzer and software (Cellular Technologies, Cleveland, Ohio).

### Preparation of Monocyte-Derived DC

Monocyte-derived DC were generated from PBMC as described previously (Nishikawa H et al., J. Immunol., 2006; 176:6340-6346; Nagata Y et al., Proc Natl Acad Sci USA., 2002; 99:10629-10634). Briefly, CD14⁺ monocytes were enriched by positive selection using CD14 Microbeads (Miltenyi Biotech) according to the manufacture's instruction. Monocytes were cultured in the presence of 1,000 U/ml GM-CSF (Immunex) and 20 ng/ml IL-4 (R&D systems) in X-VIVO-15 serum free medium (CAMBREX, Walkersville, Md.). Medium was replaced by fresh medium containing cytokines three days later. On day 6, DC were harvested and pulsed overnight with 10 μM peptide or 25 μg/ml protein.

### CFSE Labeling

Pre-sensitized CD4⁺ T cells were labeled for 7 minutes at 37° C. with 1 mM CFSE (Molecular Probes, Eugene, Oreg.) and were washed 3 times with RPMI 1640 containing 10% fetal bovine serum. CFSE-labeled CD4⁺ T cells were cultured with irradiated CD3-depleted PBMC pulsed with 10 μM peptide for 6 days. CFSE-labeled cells were analyzed for fluorescence intensity using a flow cytometer (FACSCalibur BD Biosciences).

### Proliferation Assay

5×10⁴ pre-sensitized T cells were cultured in the presence or absence of 5×10⁴ regulatory T cells with 5×10⁴ irradiated CD3-depleted PBMC pulsed with 10 μM of peptides. CD4⁺ T cells were enriched from PBMC by negative selection using CD4⁺ Isolation Kit (Miltenyi Biotec) followed by flow cytometry sorting of CD4⁺CD25^{high} T cells (FACSAria, BD Biosciences). These CD4⁺CD25^{high} cells were stimulated with 1 μg/ml anti-CD3 mAb coated plates for three days with 10 U/ml IL-2 and used as regulatory T cells. Proliferation was evaluated by pulsing cells with 1 μCi/well ³H thymidine for the last 18 hours of 72 hour culture. ³H thymidine incorporation was measured by a scintillation counter.

### Statistical Analysis

Statistical analyses were performed with the Student's paired t test. Values of p<0.05 were considered significant.

### Example 2

### NY-ESO-1-Specific CD4⁺ T Cells are Elicited by S. typhimurium-NY-ESO-1 without CD4⁺CD25⁺ T Cell Depletion

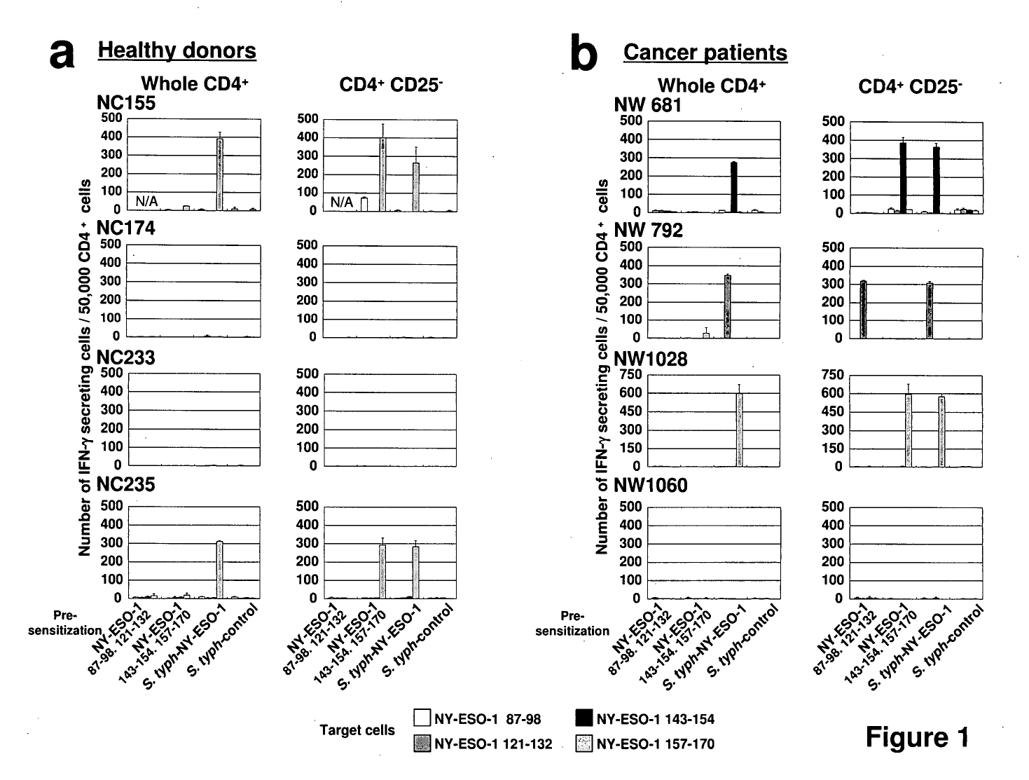

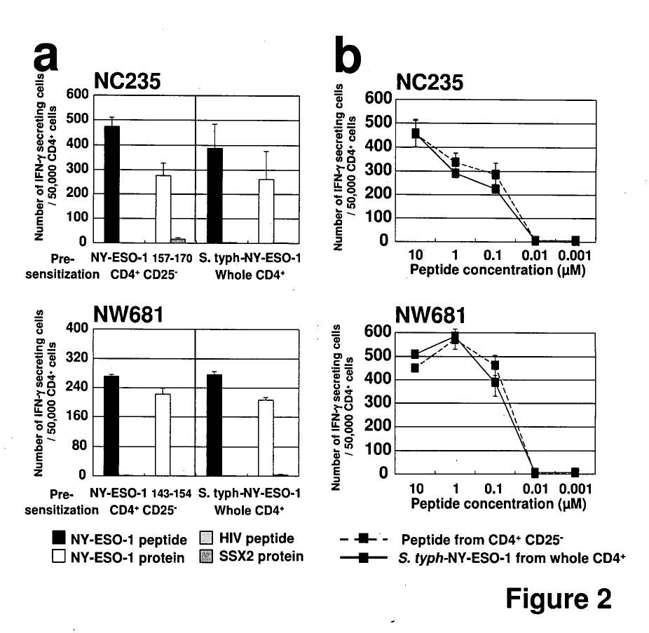

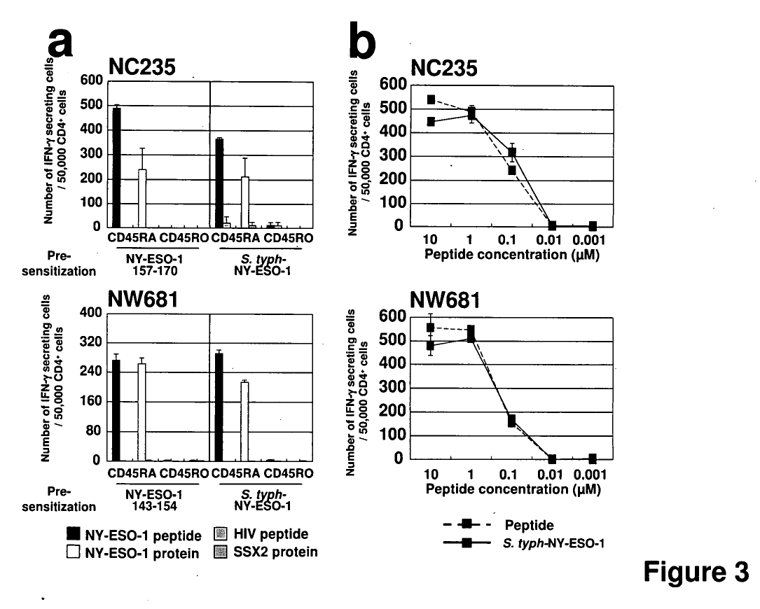

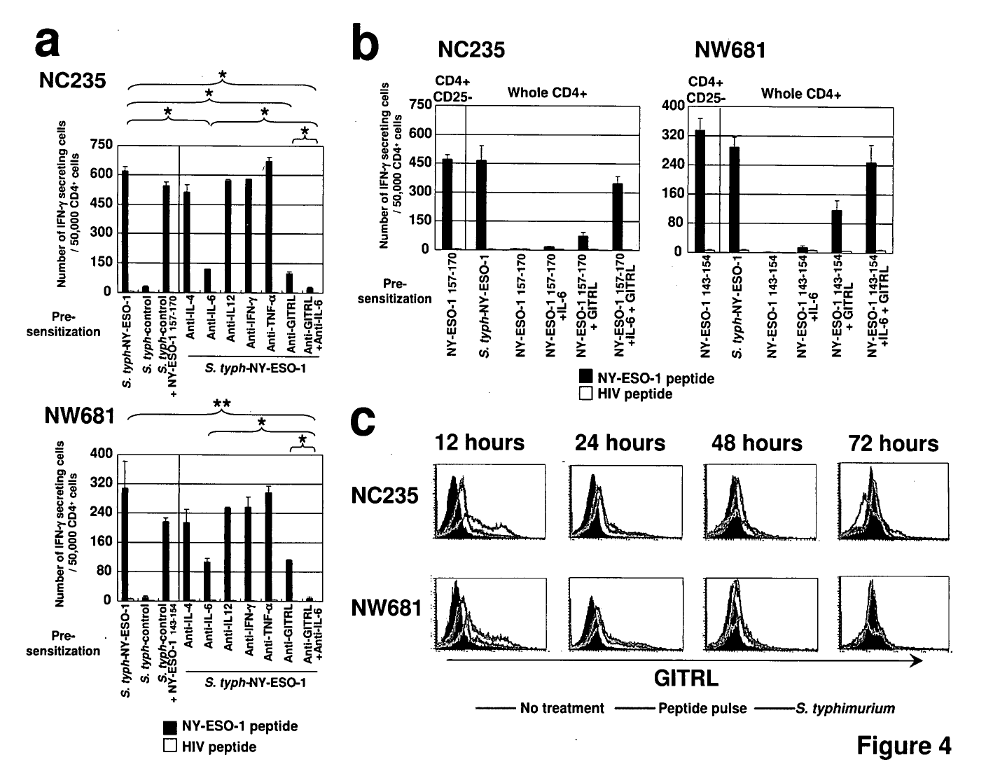

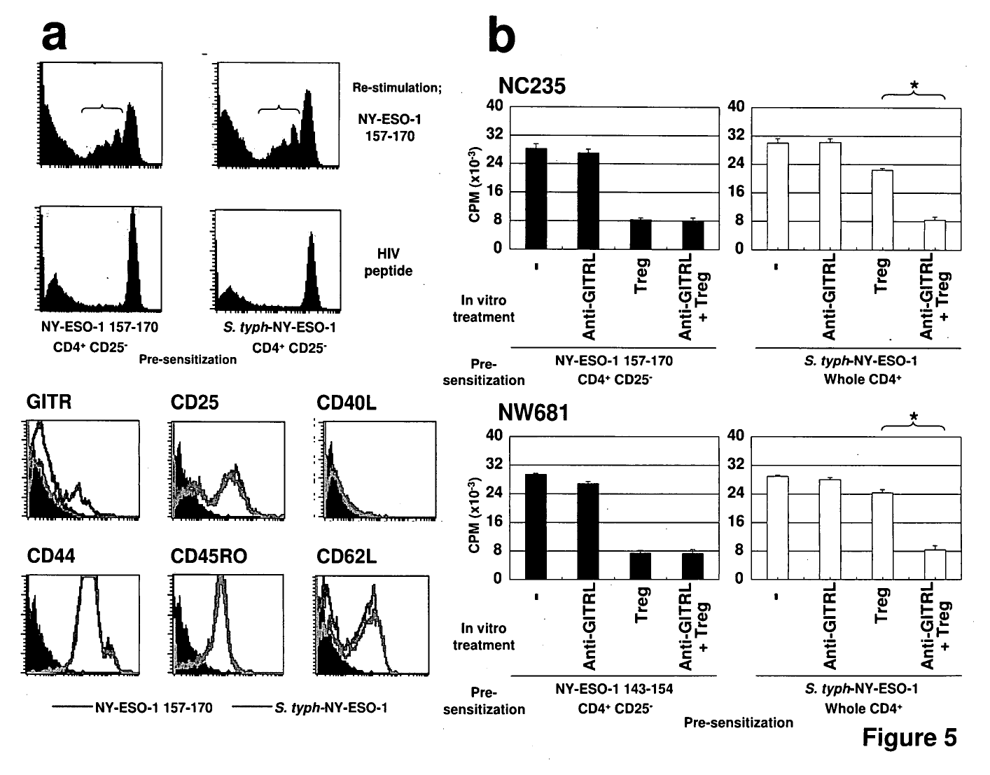

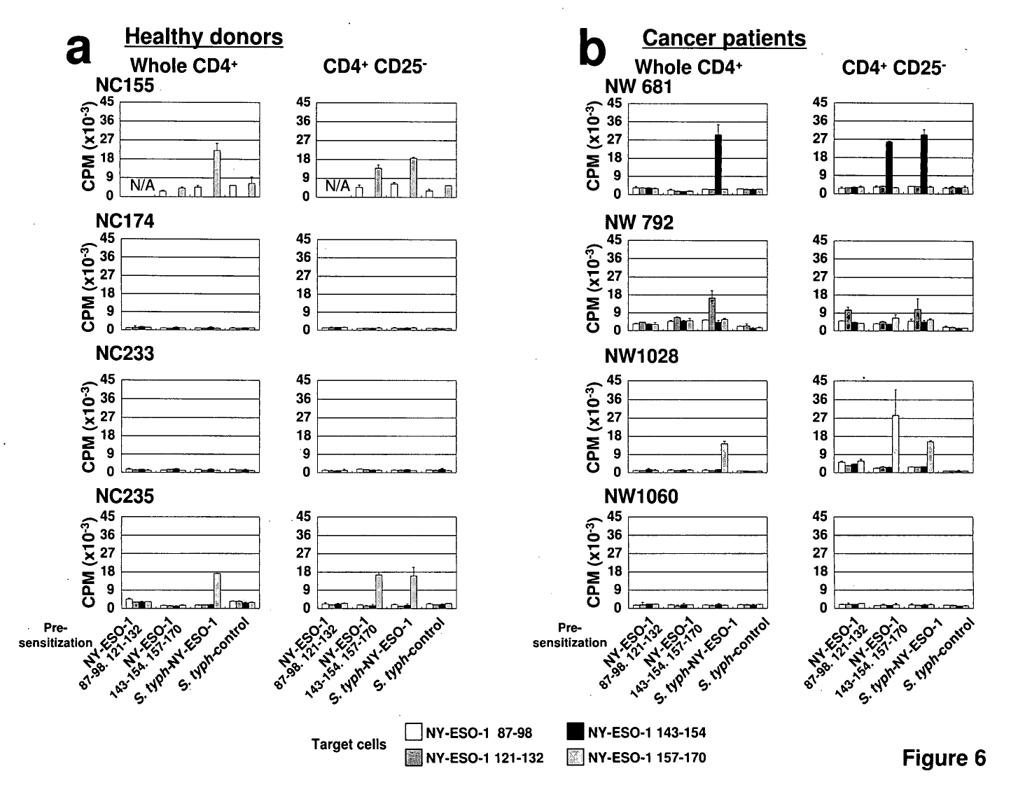

CD4⁺ T cells and CD4⁺CD25⁻ T cells were isolated from peripheral blood mononuclear cells (PBMC) and were cultured with antigen presenting cells (APC) pulsed with a series of HLA class II restricted NY-ESO-1 peptides or infected with S. typhimurium-NY-ESO-1, a S. typhimurium ΔphoP ΔphoQ avirulent strain carrying a plasmid that expresses a chimeric protein composed of the first 104 amino acids of the type III secreted protein SopE fused to reporter epitopes and the entire amino acid sequence of the NY-ESO-1 tumor antigen, or with a S. typhimurium control strain carrying an equivalent plasmid that expresses only the first 104 amino acids of the type III secreted protein SopE and reporter epitopes but not NY-ESO-1, as reported previously (Nishikawa H et al., J Clin Invest., 2006; 116:1946-1954; Nishikawa H et al., Blood., 2005; 106:1008-1011). Fifteen to 20 days later, peptide-specific interferon (IFN)-γ secreting CD4+ T helper type 1 T (Th1) cell induction was analyzed by enzyme-linked immunospot (ELISPOT) and proliferation assays. In accordance with our previous reports using NY-ESO-1 peptide-pulsed APC (Nishikawa H et al., Blood., 2005; 106:1008-1011 Nishikawa H et al., J. Immunol., 2006; 176:6340-6346), specific Th1 cells were elicited in a proportion of healthy donors and cancer patients seronegative for NY-ESO-1, but only after CD4+CD25+ T cell depletion (FIG. 1 and FIG. 6). In contrast, when using APC infected with S. typhimurium-NY-ESO-1, specific Th1 cells were elicited from whole CD4⁺ T cells without the need for CD4⁺CD25⁺ T cell depletion (FIG. 1 and FIG. 6). This specific T cell induction was dependent on NY-ESO-1 antigen delivery by S. typhimurium because the S. typhimurium control strain was not able to induce NY-ESO-1 peptide-specific CD4⁺ Th1 cells (FIG. 1 and FIG. 6). Interestingly, S. typhimurium-NY-ESO-1 only elicited Th1 responses in healthy donors and patients for whom Th1 cells were also elicited by peptide-pulsed APC following CD4⁺CD25⁺ T cell depletion, namely having NY-ESO-1-specific CD4⁺ T cell precursors (FIG. 1 and FIG. 6). Because of limitations in sample amounts, we selected two typical donors, NC235 and NW681, for further studies with healthy donors and patients with NY-ESO-1 expressing tumors but seronegative for NY-ESO-1, respectively.

### Example 3

### S. typhimurium-Induced NY-ESO-1-specific CD4⁺ Th1 Cells are Able to Recognize Naturally Processed NY-ESO-1 Protein

Our previous data have suggested that synthetic peptide vaccination induces CD4⁺ T cells with lower avidity than preexisting NY-ESO-1-specific CD4⁺ T cell precursors and spontaneously-induced CD4+ T cells (Gnjatic S et al., Adv Cancer Res., 2006; 95:1-30; Nishikawa H et al., Blood., 2005; 106:1008-1011; Nishikawa H et al., J Immunol., 2006; 176:6340-6346). For this reason, it is interesting to investigate the avidity of CD4+ T cells induced by S. typhimurium. First, we assessed whether S. typhimurium-induced NY-ESO-1-specific CD4⁺ Th1 cells were able to recognize naturally processed NY-ESO-1 protein. Whole CD4⁺ T cells derived from healthy donor NC235 and melanoma patient NW681 were presensitized with S. typhimurium-infected APC and analyzed for their capacity to recognize naturally processed antigen by ELISPOT assay using protein-pulsed autologous dendritic cells (DC) as APC. As positive control, we confirmed that NY-ESO-1-specific CD4⁺ Th1 cells derived from CD4⁺CD25⁻ T cells and presensitized with APC pulsed with NY-ESO-1 peptides had high-avidity and were able to recognize NY-ESO-1 protein-pulsed DC (FIG. 1), as previously reported (Nishikawa H et al., J Immunol., 2006; 176:6340-6346). Remarkably, NY-ESO-1-specific CD4⁺ Th1 cells induced by S. typhimurium-NY-ESO-1 from whole CD4⁺ T cells, without the need for CD4⁺CD25⁺ T depletion, were similarly able to recognize both NY-ESO-1 peptide-pulsed and protein-pulsed DC (FIG. 2a). Next, specific IFN-γ secretion by these cells was analyzed against PHA-activated CD4⁺ T cells (T-APC) pulsed with serial dilutions of peptides by ELISPOT assay. S. typhimurium-induced Th1 cells derived from whole CD4⁺ T cells and peptide-induced Th1 cells derived from CD4⁺CD25⁻ T cells were both high-avidity, and could recognize as little as 0.1 μM of peptide (FIG. 2b).

### Example 4

### S. typhimurium-NY-ESO-1 Activates High-Avidity NY-ESO-1-Specific CD4⁺ T Cell Precursors from Naïve T Cell Population

We have reported that high-avidity NY-ESO-1-specific CD4⁺ T cell precursors are present in naïve CD45RA⁺ populations and that their activation is highly suppressed by CD4⁺CD25⁺ Tregs (Gnjatic S et al., Adv Cancer Res., 2006; 95: 1-30; Nishikawa H et al., Blood., 2005; 106:1008-1011; Nishikawa H et al., J Immunol., 2006; 176:6340-6346). We investigated whether S. typhimurium also would elicit NY-ESO-1-specific CD4⁺ Th1 cells by activating high-avidity NY-ESO-1-specific CD4⁺ T cell precursors from CD45RA+ populations. CD4⁺CD25⁻ T cells were further separated into naïve and effector/memory populations according to typical surface marker molecules CD45RA and CD45RO, respectively (Nishikawa H et al., Blood., 2005; 106:1008-1011). NY-ESO-1-specific Th1 cells were exclusively elicited by S. typhimurium-NY-ESO-1 from CD4⁺CD25⁻ CD45RA⁺ naïve precursors, but not from CD4⁺CD25⁻ CD45RO⁺ memory populations of NC235 and NW681. Similar results were obtained in experiments using separated naïve and memory populations from whole CD4⁺ T cells, without depletion of CD25⁺ T cells (data not shown). These NY-ESO-1-specific CD4⁺ Th1 cells were able to recognize naturally processed protein antigen. They also had high-avidity and could recognize as little as 0.1 μM of peptide, similarly to peptide-induced Th1 cells derived from CD4⁺CD25⁻ T cells (FIG. 3).

Taken together, these data suggest that S. typhimurium-NY-ESO-1 is able to elicit NY-ESO-1-specific high-avidity CD4⁺ Th1 cells in the presence of CD4⁺CD25⁺ Tregs from NY-ESO-1-specific preexisting naïve CD4⁺ T cell precursors.

### Example 5

### IL-6 Induced by Infection of S. typhimurium is Required for Eliciting NY-ESO-1-Specific CD4⁺ Th1 Cells

We next asked whether the infection signal of S. typhimurium was required to elicit NY-ESO-1-specific CD4⁺ Th1 cells cells in the presence of CD4⁺CD25⁺ Tregs. To address this question, APC infected with a S. typhimurium control strain, identical to S. typhimurium-NY-ESO-1 but lacking expression of NY-ESO-1 protein, were pulsed with NY-ESO-1 peptide and used to stimulate whole CD4⁺ T cells. As shown in FIG. 4a, NY-ESO-1-specific CD4⁺ Th1 cells were elicited by combining S. typhimurium control strain infection and NY-ESO-1 peptide pulse. In contrast, S. typhimurium control strain alone was not able to elicit NY-ESO-1-specific CD4⁺ Th1 cells.

Since the signal provided by S. typhimurium infection is sufficient to bypass CD4⁺CD25⁺ Treg suppression and uncover NY-ESO-1-specific Th1 cell precursors, we explored the requirement of several cytokines that could be produced by S. typhimurium infection in this process using monoclonal antibody blocking. Among cytokines examined, only blocking of IL-6 partially inhibited the elicitation of NY-ESO-1-specific Th1 cells (FIG. 4a) (NC235; p<0.01, NW681; p=0.06). However, IL-6 signaling alone was not sufficient to bypass Treg suppression, since no NY-ESO-1 peptide-specific Th1 cells could be detected from whole CD4⁺ T cells by peptide-pulsed APC in the presence of recombinant IL-6 (FIG. 4b). These data suggest that other mechanism(s) also contribute to the Treg counteracting capacity of S. typhimurium.

### Example 6

### GITR Signal is Also Required for Induction of NY-ESO-1-Specific CD4⁺ Th1 cells by S. typhimurium

Recently, it has been reported that GITR signaling is important to overcome suppression of CD4⁺CD25⁺ Tregs and GITR-ligand (GITRL) cell surface expression is up-regulated after viral infection (Stephens G L et al., J Immunol., 2004; 173:5008-5020; Suvas S et al., J Virol., 2005; 79:11935-11942). We first examined the kinetics of GITRL expression following S. typhimurium infection. As shown in FIG. 4c, the expression level of GITRL on APC used for presensitization was upregulated after S. typhimurium infection. The expression was the highest after 12 hours and decreased thereafter to lower levels than the basal expression. As predicted, blocking the GITR signal using anti-GITRL antibody partially inhibited the elicitation of NY-ESO-1-specific CD4⁺ Th1 cells (FIG. 4a) (NC235; p<0.01, NW681; p=0.06). Furthermore, the presence of both anti-GITRL and anti-IL-6 blocking antibodies completely inhibited the elicitation of NY-ESO-1-specific CD4⁺ Th1 cells by S. typhimurium (FIG. 4a) (NC235; p<0.01, NW681; p<0.05). Alternatively, providing exogenous GITR signaling with a fusion GITRL-Fc protein was able to partially overcome Treg suppression since NY-ESO-1 peptide-specific CD4⁺ Th1 cells were elicited from whole CD4⁺ T cells by peptide-pulsed APC in the presence of GITRL-Fc (FIG. 4b). Induction of NY-ESO-1 peptide-specific CD4⁺ Th1 cells from whole CD4⁺ T cells by peptide-pulsed APC was further enhanced in the presence of both GITRL-Fc and IL-6. These data suggest that IL-6 and GITR signals act independently and synergistically to overcome the suppression of CD4⁺CD25⁺ Tregs.

### Example 7

### S. typhimurium-induced CD4⁺ Th1 cells are resistant against suppression of CD4⁺CD25+Tregs

We next examined potential differences between CD4⁺ Th1 cells induced by NY-ESO-1 peptide after CD4⁺CD25⁺ T cell depletion and S. typhimurium-induced CD4⁺ Th1 cells. To minimize differences in the procedures and avoid potential influence of CD4⁺CD25⁺ Tregs on the phenotype of responder cells, both peptide-induced CD4⁺ Th1 cells and S. typhimurium-induced CD4⁺ Th1 cells were generated from CD4⁺CD25⁻ T cell populations. To identify NY-ESO-1-specific CD4⁺ T cells, presensitized CD4⁺ T cells were labeled with carboxyfluorescein diacetate succinimidyl ester (CFSE) and restimulated with NY-ESO-1 or control peptides. Cells showing CFSE dilution were considered to contain NY-ESO-1-specific CD4⁺ T cells and were gated. Among surface activation markers tested, only GITR expression was higher on S. typhimurium-induced CD4⁺ Th1 cells than on peptide-induced CD4⁺ Th1 cells, while other surface activation markers were similarly expressed on specific Th1 cells by both induction methods (FIG. 5a). This up-regulation of GITR expression was also observed when S. typhimurium-induced CD4⁺ Th1 cells were elicited from whole CD4⁺ T cells (data not shown).

The capacity of S. typhimurium-NY-ESO-1 to elicit NY-ESO-1-specific high-avidity CD4⁺ Th1 cells in the presence of CD4⁺CD25⁺ Tregs prompted us to examine whether S. typhimurium-induced CD4⁺ Th1 cells were resistant to CD4⁺CD25⁺ Tregs. To maintain antigen specificity of CD4⁺ Th1 cells, we avoided using anti-CD3 antibody for in vitro CD4⁺CD25⁺ Treg activation during the coculture with effector cells. Rather, CD4⁺CD25+Tregs were isolated from PBMC and preactivated with anti-CD3 antibody-coated plates for three days. Then, CD4⁺CD25⁻ or CD4⁺ T cells presensitized with peptide or S. typhimurium-NY-ESO-1, respectively, were cocultured with T cell-depleted PBMC pulsed with NY-ESO-1 peptides in the presence or absence of preactivated Tregs. These Tregs showed suppressive activity against NY-ESO-1-specific proliferation of peptide-induced Th1 cells (FIG. 5b). In contrast, S. typhimurium-induced CD4⁺ Th1 cells were able to proliferate in the presence of preactivated Tregs (FIG. 5b), thus displaying relative resistance to Tregs. Since expression levels of GITR significantly differed between NY-ESO-1 peptide-induced and S. typhimurium-induced CD4⁺ Th1, the effect of blocking GITR signaling was assessed by anti-GITRL antibody (antagonistic anti-GITR blocking reagents are not yet available). As shown in FIG. 5b, the treatment completely abrogated the resistance to CD4⁺CD25⁺ Tregs of S. typhimurium-induced CD4⁺ Th1 cells (NC235; p<0.01, NW681; p=0.01).

### Example 8

### Induction of Regulatory T Cell-Resistant Helper CD4⁺ T Cells

We have recently engineered a recombinant S. typhimurium-NY-ESO-1 strain that uses a specialized type III protein secretion system to inject bacterially-produced NY-ESO-1 protein into host cells (Nishikawa H et al., J Clin Invest., 2006; 116:1946-1954). This strain could elicit NY-ESO-1-specific CD8⁺ and CD4⁺ T cell responses in vitro in cancer patients with NY-ES 0-1 spontaneous immunity and eradicate NY-ES 0-1 expressing tumors in vivo in mice (Nishikawa H et al., J Clin Invest., 2006; 116:1946-1954). In the current study, we examined the capacity of this construct to influence the suppressive activity of CD4⁺CD25⁺ Tregs and elicit NY-ESO-1-specific CD4⁺ Th1 cells resistant to Treg suppression. We found that S. typhimurium-NY-ESO-1 could elicit NY-ESO-1-specific CD4⁺ Th1 cells in vitro in healthy donors as well as in melanoma patients with NY-ESO-1 expressing tumors but without spontaneous NY-ESO-1 immunity. Stimulation with S. typhimurium-NY-ESO-1 was able to elicit NY-ESO-1-specific CD4⁺ Th1 cells in these patients in the presence of CD4⁺CD25⁺ Tregs, while peptide stimulation required prior depletion of CD4⁺CD25⁺ Tregs to do so (FIG. 1 and FIG. 6). Furthermore, S. typhimurium-NY-ESO-1 elicited NY-ESO-1-specific CD4⁺ Th1 cells from high-avidity NY-ESO-1-specific naïve CD4⁺ T cell precursors (FIGS. 2, 3), which are normally kept under CD4⁺CD25⁺ Treg tight control (Nishikawa H et al., J Immunol., 2006; 176:6340-6346). As a result, S. typhimurium-induced NY-ESO-1-specific CD4⁺ Th1 cells had high-avidity and recognized naturally processed protein antigen as efficiently as CD4⁺ Th1 cells elicited from patients with NY-ESO-1 spontaneous immunity. These data suggest that S. typhimurium-NY-ESO-1 can elicit NY-ESO-1-specific CD4⁺ Th1 cells by inhibiting or resisting the suppressive activity of CD4⁺CD25⁺ Tregs. These S. typhimurium-induced NY-ESO-1-specific CD4⁺ Th1 cells could therefore be closer to CD4⁺ Th1 readily observed in patients with NY-ESO-1 spontaneous immunity, where they may contribute to B cell activation for establishing specific antibody and help CD8⁺ T cell induction (Gnjatic S et al., Adv Cancer Res., 2006; 95:1-30; Jäger E et al., J Exp Med., 1998; 187:265-270; Jäger E et al., Proc Natl Acad Sci USA., 2000; 97:4760-4765; Gnjatic S et al., Proc Natl Acad Sci USA., 2003; 100:8862-8867).

Our data also showed that induction of NY-ESO-1-specific CD4⁺ Th1 cells by S. typhimurium-NY-ESO-1 was strictly limited to cancer patients and healthy donors with high-avidity NY-ESO-1-specific naïve CD4⁺ T cell precursors (FIG. 1 and FIG. 6). These data suggest that we may predict which individuals are better responders to vaccination with S. typhimurium-NY-ESO-1 and elicit NY-ESO-1-specific CD4⁺ T cells, using in vitro assays to examine the presence of high-avidity NY-ESO-1-specific naïve CD4⁺ T cell precursors.

The capacity of S. typhimurium to overcome the suppressive activity of CD4⁺CD25⁺ Tregs was dependent on IL-6 but not on other inflammatory cytokines tested (FIG. 4a). IL-6, a proinflammatory cytokine that plays an important role in host defense against infections, is also considered to have an important role for the inhibition of CD4⁺CD25⁺ Tregs (Pasare C et al., Science., 2003; 299:1033-1036; Doganci A et al., J Clin Invest., 2005; 115:313-325). IL-6 secretion from APC following bacterial infection, including S. typhimurium infection, is considered to be the consequence of TLR stimulation by pathogen associated molecules such as lipopolysaccharide (Iwasaki A et al., Nat. Immunol., 2004; 5:987-995; Akira S et al., Nat Rev Immunol., 2004; 4:499-511). Our results are in accordance with a previous report showing that IL-6 is critical for overcoming suppression of CD4⁺CD25⁺ Tregs via TLR signaling (Pasare C et al., Science., 2003; 299:1033-1036). However, other mechanisms were also predicted from our data to be involved in the capacity of S. typhimurium to control Treg suppression since; 1) blocking by anti-IL-6 antibody only had a partial effect (FIG. 4a); and 2) addition of recombinant IL-6 alone was not sufficient to elicit NY-ESO-1 peptide-specific CD4⁺ Th1 cells in the presence of CD4⁺CD25⁺ Tregs (FIG. 4b). Therefore, it is possible that responses stimulated by S. typhimurium other than those dependent on TLR receptors may also influence its ability to suppress CD4⁺CD25⁺ Tregs. For example, S. typhimurium is capable of inducing a significant reprogramming of gene expression in infected cells in a manner that is dependent on the function of one of its type III secretion systems (Galán J E, Annu Rev Cell Dev Biol., 2001; 17:53-86).

Disrupting GITR signaling using anti-GITRL blocking antibody also decreased the capacity of S. typhimurium to overcome the suppressive activity of CD4⁺CD25⁺ Tregs (FIG. 4a). As previously reported for virus infections and TLR signaling (Stephens G L et al., J Immunol., 2004; 173:5008-5020; Suvas S et al., J Virol., 2005; 79:11935-11942), we observed that GITRL expression on APC was upregulated after S. typhimurium infection (FIG. 4c). It was shown using GITR knockout mice that T cell mediated suppression is abrogated by GITR engagement on CD4⁺CD25⁻ effector T cells, but not CD4⁺CD25⁺ Tregs as originally thought (Stephens G L et al., J Immunol., 2004; 173:5008-5020; Shimizu J et al., Nat. Immunol., 2002; 3:135-142; Shevach E M et al., Nat Rev Immunol., 2006; 6:613-618). It is plausible that S. typhimurium-induced GITRL on APC engages with GITR on activated effector T cells and make them resistant to suppression by CD4⁺CD25⁺ Tregs. This hypothesis is supported by the following data; 1) S. typhimurium-induced CD4⁺ Th1 cells were able to proliferate in the presence of activated CD4⁺CD25⁺ Tregs (FIG. 5b); 2) GITR expression was maintained in S. typhimurium-induced CD4⁺ Th1 cells, but not peptide-induced CD4⁺ Th1 cells (FIG. 5a); and 3) addition of GITRL-Fc had a partial effect on the induction of NY-ESO-1 peptide-specific Th1 response in the presence of Tregs (FIG. 4b). Recently, it has been reported that flagellin, a TLR5 ligand present in Salmonella, can directly enhance the suppressive capacity of CD4⁺CD25⁺ Tregs but has an opposite indirect effect in the presence of APC where it overrides suppression (Stecher B et al., Infect Immun., 2004; 72:4138-4150; Crellin N K et al., J Immunol., 2005; 175:8051-8059). In our study, while a direct effect S. typhimurium on Tregs cannot be excluded, one mechanism for overcoming suppressive activity is more likely to be the engagement of GITR on responder effector cells rather than on CD4⁺CD25⁺ Tregs themselves, because addition of blocking anti-GITRL antibody did not enhance the suppressive activity of CD4⁺CD25⁺ Tregs for peptide-induced CD4⁺ Th1 cells (FIG. 5b).

Although both anti-IL-6 and anti-GITRL antibodies blocked the capacity of S. typhimurium to inhibit the suppressive activity of CD4⁺CD25⁺ Tregs, these molecules seem to exploit different mechanisms since; 1) addition of both anti-IL-6 and anti-GITRL antibodies worked together to completely abrogate the effect of S. typhimurium (FIG. 4a); and 2) addition of an agonistic GITRL fusion molecule was able to elicit NY-ESO-1-specific Th1 cells from whole CD4⁺ T cells without the need for depletion of CD4⁺CD25⁺ Tregs whereas addition of IL-6 alone could not (FIG. 4c). Furthermore, in the restimulation phase, enhanced GITR expression was observed on Treg-resistant S. typhimurium-induced CD4⁺ Th1 cells, but not on Treg-sensitive peptide-induced CD4⁺ Th1 cells, and GITR signal alone was sufficient to overcome the suppressive activity of CD4⁺CD25⁺ Tregs (FIG. 5). The question remains whether anti-IL-6 or anti-GITRL antibody treatment affects the inhibition of Th1 induction differently in normal volunteers and in melanoma patients. It is possible that a difference exists between individuals or between normal individuals and cancer patients in the sensitivity of their Tregs to IL-6 and/or GITR ligand. Taken together, it is considered that IL-6 and GITR signaling synergistically act to overcome suppression by CD4⁺CD25⁺ Tregs in the priming phase and to make effector cells resistant to CD4⁺CD25⁺ Tregs. In the restimulation phase, GITR signaling is sufficient to block the suppressive activity of CD4⁺CD25⁺ Tregs.

We show here an additional potential of GITR-GITRL signal as a critical molecules for Treg resistance. The co-stimulatory activity of GITR-GITRL signal is still under debate. It has recently been reported that several human tumor cell lines express substantial levels of GITRL and that this constitutive expression diminishes NK cell activity against tumor cells (Baltz K M et al., Faseb J., 2007). On the other hand, it was also shown that plasmacytoid DC activated with CpG promote NK cell activity through GITRL (Hanabuchi S et al., Blood., 2006; 107:3617-3623). Several reports propose that co-stimulatory or inhibitory effects are dependent on strength of antigen stimulation, type of target cells (CD4⁺ or CD8⁺ T cells) or sum of other co-stimulatory signals (Kanamaru F et al., J Immunol., 2004; 172:7306-7314; Tone M et al., Proc Natl Acad Sci USA., 2003; 100:15059-15064; Muriglan S J et al., J Exp Med., 2004; 200:149-157; Ronchetti S et al., Eur J Immunol., 2004; 34:613-622). The cell types expressing GITRL may also be a critical factor. Further studies appear necessary for clinical application of this molecule.

Our strategy to overcome the suppressive function of CD4⁺CD25⁺ Tregs using S. typhimurium has an advantage compared to other methods such as eliminating CD4⁺CD25+Tregs. It has been reported that downregulation of CD4⁺CD25⁺ Treg activity and blocking of non-specific suppressive mechanisms are accompanied by autoimmune diseases (Shimizu J et al., J Immunol., 1999; 163:5211-5218; Hodi F S et al., Proc Natl Acad Sci USA., 2003; 100:4712-4717; Phan G Q et al., Proc Natl Acad Sci USA., 2003; 100:8372-8377). Since S. typhimurium-induced Treg-resistant Th1 cells are directed to a tumor antigen not expressed in normal somatic tissues, occurrence of autoimmune diseases is less likely. We propose that S. typhimurium is a promising cancer vaccine platform for specific immunotherapy that could surmount the obstacle of CD4⁺CD25⁺ Treg activity.

### Example 9

### Prediction of Tumor Vaccination Response Outcomes

S. typhimurium-NY-ESO-1 elicits Th1 responses in individuals for whom Th1 cells were also elicited by peptide-pulsed APC following CD4⁺CD25⁺ T cell depletion, namely having NY-ESO-1-specific CD4⁺ T cell precursors (FIGS. 1 and 6). Thus, induction of NY-ESO-1-specific CD4⁺ Th1 cells by S. typhimurium-NY-ESO-1 is limited to individuals with high-avidity NY-ESO-1-specific naïve CD4⁺ T cell precursors (FIG. 1 and FIG. 6).

Peripheral blood samples or other biological samples that contain T cell precursors are obtained from individual(s) having, or suspected of having cancer. These samples are used for in vitro assays to analyze the presence of antigen specific naïve CD4⁺ T cell precursors (particularly cancer antigens, e.g., NY-ESO-1). A prediction of tumor vaccination response outcome is made based on the results of this analysis. Individual(s) who have in their samples antigen-specific naïve CD4⁺ T cell precursors, particularly high-avidity precursors, are predicted to respond to vaccination with avirulent bacteria and elicit antigen-specific CD4⁺ T cells.

Prediction in this manner is not limited to tumor vaccination with a bacterium comprising a cancer testis antigen. Rather, prediction can be made using any of the vaccination methods, disclosed herein, that are associated with regulatory resistant helper CD4⁺ T cell induction. For example, vaccination may be through the delivery of a soluble antigen peptide and avirulent gram-negative bacterium (e.g., S. typhimurium) that does itself not comprise the antigen.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for producing antigen-specific CD4+ T cells in the presence of CD4+CD25+ T regulatory cells comprising
contacting a population of CD4+ precursor cells with antigen presenting cells, wherein the antigen presenting cells have been contacted with glucocorticoid-induced TNF receptor ligand (GITRL), and a polypeptide antigen or an immunogenic fragment thereof,
wherein the population of CD4+ precursor cells is not depleted of CD4+CD25+ T regulatory cells, and
whereby the polypeptide antigen or the immunogenic fragment thereof stimulates production of antigen-specific CD4+ T cells specific for the polypeptide antigen or the immunogenic fragment thereof.

2. The method of claim 1, wherein the step of contacting a population of CD4+ precursor cells comprises administering the GITRL and the polypeptide antigen or the immunogenic fragment thereof to a subject in need of such treatment, in amounts of each that are effective to stimulate production of antigen-specific CD4+ T cells.

3. The method of claim 1, wherein the CD4+ precursor cells are peripheral blood mononuclear cells.

4. The method of claim 1, further comprising isolating the antigen-specific CD4+ T cells.

5. The method of claim 1, wherein the polypeptide antigen or immunogenic fragment thereof is a tumor antigen protein or an immunogenic fragment thereof.

6. The method of claim 5, wherein the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

7. The method of claim 1, wherein the antigen presenting cells are contacted with more than one polypeptide antigen or immunogenic fragment thereof.

8. The method of claim 1, wherein the antigen-specific CD4+ T cells are T helper 1 (Th1) cells.

9. The method of claim 1, further comprising contacting the antigen presenting cells with interleukin-6 (IL-6).

10. The method of claim 1, wherein the GITRL is a recombinant GITRL-Fc fusion protein.

11. The method of claim 1, wherein the antigen-specific CD4+ T cells are resistant to anti-proliferative effects of CD4+CD25+ T regulatory cells.

12. The method of claim 1, wherein the antigen-specific CD4+ T cells are activated high-avidity antigen-specific CD4+ T cell precursors from a CD45RA+ population.

13. A method for passive immunization comprising
administering to a subject in need of such treatment a population of antigen-specific CD4+ T cells as claimed in claim 1.

14. A method for preparing antigen presenting cells from peripheral blood mononuclear cells, comprising
obtaining peripheral blood mononuclear cells (PBMCs) from a subject, wherein the PBMCs are not depleted of CD4+CD25+ T regulatory cells,
contacting the PBMCs with a glucocorticoid-induced TNF receptor ligand (GITRL) and optionally with interleukin-6 (IL-6), and an antigen,
culturing the contacted PBMCs, and
isolating antigen presenting cells.

15. The method of claim 14, wherein the GITRL is a recombinant GITRL-Fc fusion protein.

16. The method of claim 14, wherein the antigen is a tumor antigen.

17. The method of claim 16, wherein the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

18. An isolated population of antigen presenting cells prepared by the method of claim 14.

19. A method for preparing antigen-specific T cells comprising
obtaining peripheral blood mononuclear cells (PBMCs) from a subject,
contacting the PBMCs with the antigen presenting cells of claim 18,
culturing the contacted PBMCs, and
isolating antigen-specific T cells from the PBMCs.

20. The method of claim 19, wherein the antigen-specific T cells are CD4⁺ T cells.

21. An isolated population of antigen-specific T cells prepared by the method of claim 19.

22. The isolated population of T cells of claim 21, wherein the T cells are CD4⁺ T cells.

23. A method for passive immunization comprising
administering to a subject in need of such treatment a population of antigen-specific T cells as claimed in claim 21.

24. A kit for immunization comprising
a first container containing one or more doses of a vaccine against an antigen,
a second container containing an amount of glucocorticoid-induced TNF receptor ligand (GITRL), and
a third container containing an amount of interleukin-6 (IL-6).

25. The kit of claim 24, wherein the antigen is not a tumor antigen.

26. The kit of claim 25, wherein the antigen causes mumps, measles, rubella, chicken pox, influenza, diphtheria, tetanus, pertussis, hepatitis A, hepatitis B, bacterial meningitis (Haemophilus influenzae type b), polio or Streptococcus pneumoniae infection (invasive pneumococcal disease).

27. The kit of claim 24, wherein the antigen is a tumor antigen.

28. The kit of claim 27, wherein the tumor antigen is NY-ESO-1, a MAGE antigen, a SSX antigen, SCP1, CT7, NY-CO-58, a BAGE antigen, a GAGE antigen, Melan-A/MART-1, gp100 or gp75.

29. The kit of claim 24, wherein the vaccine against the antigen is a DNA vaccine vector encoding the antigen.

30. The kit of claim 24, wherein the GITRL is a recombinant GITRL-Fc fusion protein.
